(19) United States
(12) Patent Application Publication
(10) Pub. No.: US 2011/0270220 A1
(43) Pub. Date: Nov. 3, 2011

(54) LINEARLY ACTUATED DISPENSER AND THERAPEUTIC PACKAGE SUITABLE FOR ADMINISTERING A THERAPEUTIC SUBSTANCE AND RELATED METHOD

(75) Inventor: Amir Genosar, Boulder, CO (US)

(73) Assignee: AKTIVPAK, INC., Boulder, CO (US)

(21) Appl. No.: 13/040,134

(22) Filed: Mar. 3, 2011

Related U.S. Application Data
(60) Provisional application No. 61/310,235, filed on Mar. 3, 2010.
(60) Provisional application No. 61/419,892, filed on Dec. 6, 2010.

(51) Int. Cl. A61M 5/31; A61M 5/32; A61M 35/00; A61J 1/14

(52) U.S. Cl. 604/506; 604/212; 604/87; 604/416; 604/500; 604/192; 604/310

(57) ABSTRACT

A device for administering a therapeutic substance includes a sealed package and an administration assembly. The sealed package includes a collapsible compartment containing the substance. The administration assembly includes a dispenser that is in fluid communication with the substance. At least one compression member is movable by linear actuation to progressively collapse the collapsible compartment and urge the therapeutic substance through the dispenser.

### CROSS-REFERENCE TO RELATED APPLICATIONS

This application claims the benefit of the filing date of U.S. Provisional Application No. 61/310,235, filed Mar. 3, 2010, and entitled LINEARLY ACTUATED DISPENSER AND THERAPEUTIC PACKAGE SUITABLE FOR ADMINISTERING A THERAPEUTIC SUBSTANCE AND RELATED METHOD, and U.S. Provisional Application No. 61/419,892, filed Dec. 6, 2010, and entitled ASEPTIC INJECTOR AND CARTRIDGE ARRANGEMENT, the disclosures of which are incorporated, in their entirety, by reference.

### TECHNICAL FIELD

The present disclosure, in one sense, relates to a dispensing package of a unit dose of therapeutic substance and administration of the therapeutic substance to a patient. More particularly the present disclosure refers to pre-filled film packages where the contents of the package are removed from the package using a linear or lateral applied force.

### BACKGROUND

Film or foil packages (generally referred here after as “film packages”) are abundant in commercial use for hermetically storing content which is sensitive to environmental condition which can cause its perish. Films can be composed to have superior barrier properties to light, air moisture and other elements encountered in and around the package. State of the art film manufacturing methods allow for combination of several monolayers in a multilayer film each providing complementary properties and qualities to the film. For example aluminum Aluminum-Oxide or Teflon laminates provide superior barrier to moisture and oxygen. Polypropylene or Polyester layer provides good formability of the film, and PE or EVA outer layers provides excellent heat sealing properties. Thus extreme properties can be incorporated in a multi-layer film without giving up on other important properties.

Achieving high barrier wall with injection molded parts (hereafter referred to as molded parts) is more challenging and relatively limited than with film walls. Molded parts are usually made from a single uniform material and where a combination of materials or properties is relatively expensive and complicated to manufacture. Also, while films are produced in a continuous process under uniform constant and controlled conditions which reduce the probability of defects in the product, injection molding cycles involve varying conditions which increase risk of defects in particular if the molded parts involve extreme properties such as very small size or very low wall thickness. Thus molded parts provide inferior barrier properties. Defects in molded parts may include cold weld lines (i.e., a seam between one segment of a part to another which is not a homogenous continuation of the material), a crack of a hole, etc. Thus molded parts provide inferior barrier properties for packaging purposes and therefore are not common in this art. Yet several sophisticated packages, which provide extra functionality, combine molded parts in a film package such that the molded part provides part of the wall of the package and therefore a barrier between the content of the pouch and the surroundings. While providing the extra functionality, the molded part is then the Achilles Heel of the hermetic sealing of the content.

U.S. Pat. No. 6,979,316 discloses an auto-injector for rapid delivery of a bolus of injectable medication. In one exemplary the auto-injector comprises a pouch reservoir where a septum is implemented at one end of the pouch for piercing the pouch with a needle. The text describes the septum to be a barrier between content of the pouch and the surrounding. The text does not support the term “septum” any further but it is assumed, based on the common terminology in the art that the inventors refer to a self-sealing compressed rubber component that can be penetrated by a sharp hollow member, such as a needle, to communicate with the content of the pouch. Common rubber materials for this application include silicon. Septums are in common use in filling sites or outlet ports of infusion bags, or other forms of containers for liquid medications.

In U.S. Pat. No. 3,554,256 a container for packaging and feeding intravenous fluids which includes a flexible tubular container member having sealed ends and an outlet connector disposed midway between the ends of the container for interconnecting with an intravenous tube. The ends of the container member are adapted to be attached to a support so that the container member can be folded over and its ends attached to the support, whereby the outlet is disposed at the bottom of the container member. At least one other connector is located near one end of the container member to permit an additive to be added to the contents of the container.

### SUMMARY

A variety of embodiments are described which broadly relate to the dispensing of a dose of a substance to a subject. In the description which follows, the term “film” refers to any thin resilient wall of a reservoir, including flexible sheets, laminated sheets or films, monolayer or multilayer, extruded, blow-molded, blown-films or calibrated (rolled) films, metal foils, etc. The term “pouch” refers to any form of reservoir at list partly constructed from films. The terms “pouches”, “blisters”, and “compartments” generally refer to packages comprising flexible walls commonly made from film, foil, extruded, blown, blow molded, stamped, cold formed, or thermoformed components.

One embodiment of a device comprises a sealed package that includes a collapsible compartment containing the substance. An administration assembly is joined to the package for relative movement there with. The administration assembly may include a delivery device that is in fluid communication with the substance. At least one compression member of the administration assembly is movable by linear actuation to progressively collapse the collapsible compartment and urge the therapeutic substance through the delivery device.

A particular embodiment provides a single use dispenser for hypothermic administration of a unit dose of a therapeutic fluid to a subject and comprises a hermetically sealed package including a collapsible first compartment containing a first therapeutic substance, with the first compartment comprising a first flexible wall, and a collapsible second compartment containing a second therapeutic substance and having an associated second flexible wall. Here, a frangible seal is disposed between the first and second compartments. A rigid backing supports the hermetically sealed package and an administration assembly is associated with the hermetically sealed package. The administration assembly includes an elongate needle in fluid communication with at least one of the first and second compartments. A compression member may be used to rupture the frangible seal such that the first and second therapeutic substances are merged. The same compression member may then be used to force the merged first and second therapeutic substances out through the needle. The compression member movement relative to the first and second components may also move the needle between extended and retracted positions. The compression member may be movable by an actuator that operates linearly.

Also described is a therapeutic package for use in administering a dose of a therapeutic substance to a subject. Such a package broadly comprises first and second sealed compartments mounted to a generally planar surface of a backing member having respective first and second walls, with each compartment pre-filled with a respective first and second therapeutic fluid. A frangible seal is disposed between the two compartments. The compartments can be merged by applying a first force to at least one of the first and second compartments in a plane generally parallel with the backing surface. The first force collapses the second compartment to cause the frangible seal to rapture such that the first and second fluids are merged. Applying a second force to the merged first and second compartments in an opposite direction that is also generally parallel with the backing surface forces the merged first and second fluids out of the therapeutic package. The force may be applied via linear actuation.

The present disclosure further discloses a compartment containing a substance. The compartment is integrally made from film such that the boundaries of the compartments are solely defined by the film, and where a dispensing assembly is manipulated to break the integrity of the package and communicate the substance with a dispensing end of the administration device. The dispensing assembly may move in opposite linear directions to first break the integrity of the package followed by dispensing the substance. Alternatively, the dispensing assembly may move in a single linear direction to first break the integrity of the package followed by dispensing the substance.

The present disclosure further discloses a prefilled dispensing device comprising an actuator operable to dispense a substance from the prefilled dispensing device. A position of the actuator before operation and after dispensing is substantially the same. The arrangement allows for compact presentation of the device during shipping and storage.

Another aspect of the present disclosure relates to a prefilled reconstitution syringe comprising an actuator operable to dispense an injectable product. The actuator is moved from a first position to a second position to mix substances of the injectable product, and is substantially returned from the second position to the first position to dispense the mixed injectable product.

The present disclosure further discloses a medical syringe for injecting a dose to a subject. The medical syringe includes a dispensing port supported by a Luer lock connector for receiving a needle. The Luer lock is retractable upon completion of injecting the dose to retract at least a portion of the needle into the medical syringe. Retracting at least a portion of the needle may protect a tip of the needle.

Another aspect of the present disclosure relates to a dispensing device that includes a sealed package comprising at least one compartment containing a substance, a backing having at least one compression surface with a first surface shape when in a rest position, and a compression panel. The compression panel is configured to compress the compartment between the backing and the compression panel. The compression panel includes at least one compression surface with a second surface shape when in a rest position that is different than the first surface shape. At least one of the first and second surface shapes is deformable to substantially match a shape of the other during compression of the at least one compartment.

The present disclosure also discloses a medical syringe for injecting a dose to a subject. The medical syringe includes a dispensing assembly supporting a Luer lock connector that is configured to receive a needle. The Luer lock is retractable upon completion of injecting the dose to draw at least a portion of the needle into the medical syringe.

The present disclosure further discloses a sealed package that includes a backing and at least one compartment mounted to the backing. The backing includes an extension accommodating a dispenser. The at least one compartment contains at least one substance. The backing may include a first surface upon which the at least one compartment is mounted, and the extension extends generally perpendicular to the first surface. The sealed package may also include a channel disposed on the backing that provides fluid communication between the at least one compartment and the administration feature.

The present disclosure discloses a dispensing device that includes a compression member and a sealed package. The sealed package comprises a first frangible seal and a second frangible seal that at least partially define a perimeter of a first compartment. Moving the compression member in a first direction ruptures the first frangible seal, and moving the compression panel in a second direction ruptures the second frangible seal.

A method is also provided for preparing to administer a dose of the therapeutic substance to a patient. According to the method an administration device is provided that includes a sealed package and an administration assembly such as described above. The administration assembly is advanced from the pre-administration position toward the ready position causing merging of first and second fluids of the sealed package to create the therapeutic substance. The administration assembly is advanced from the ready position toward a delivery position causing the therapeutic substance to be dispersed. Advancing the administration assembly may include linear or axial movement. Advancing the administration assembly may be achieved via linear actuation.

Another method relates to dispensing a substance to a subject. The method includes providing a dispensing device having a compression member, a housing, and a sealed package, wherein the sealed package includes first and second sealed compartments each containing different substances. The method further includes moving the compression member relative to the housing to apply a linear compression force to at least one of the first and second sealed compartments to merge the substances of the first and second sealed compartments, and compressing at least one of the first and second sealed compartments to dispense the merged substances to the subject.

These and other aspects of the present disclosure will become more readily appreciated and understood from a consideration of the following detailed description of the preferred embodiments when taken together with the accompanying drawings, in which:

### BRIEF DESCRIPTION OF THE DRAWINGS

FIG. 1 is a perspective view of an example delivery device in accordance with the present disclosure.

FIG. 2 is a perspective view of a sealed package of a delivery device of FIG. 1.

FIG. 3 is a cross-sectional perspective view of the delivery device of FIG. 1.

FIG. 4 is a cross-sectional perspective view of a delivery device of FIG. 1 with the administration assembly retracted to a rear end of the sealed package.

FIG. 5 is a cross-sectional perspective view of the delivery device of FIG. 4 with the administration assembly advanced partially distally to advance a needle from the housing.

FIG. 6 is a cross-sectional perspective view of the delivery device of FIG. 5 with the administration assembly advanced distally to dispense the contents of the sealed package and positioned to retract the needle into the housing.

FIG. 7 is a perspective view of another example delivery device in accordance with the present disclosure.

FIG. 8 is a top exploded perspective view of the delivery device of FIG. 7.

FIG. 9 is a bottom exploded perspective view of the delivery device of FIG. 7.

FIG. 10 is a top perspective view of another example delivery device in accordance with the present disclosure.

FIG. 11 is a top exploded perspective view of the delivery device of FIG. 10.

FIG. 12 is another top exploded perspective view of the delivery device of FIG. 10.

FIG. 13 is a bottom exploded perspective view of the delivery device of FIG. 10.

FIG. 14 is a top perspective view of another example delivery device in accordance with the present disclosure.

FIG. 15 is a top perspective view of the delivery device of FIG. 14 illustrating internal features of a delivery device.

FIG. 16 is a top perspective view of a sealed package of the delivery device of FIG. 15.

FIG. 17 is a bottom perspective view of the sealed package of FIG. 15.

FIG. 18 is a side view of the delivery device of FIG. 15.

FIG. 19 is a cross-sectional side view of the delivery device of FIG. 15 with the administration assembly in an initial distal rest state.

FIG. 20 is a cross-sectional side view of the delivery device of FIG. 19 with the administration assembly moved proximally to merge first and second sealed compartments of the sealed package.

FIG. 21 is a cross-sectional side view of the delivery device of FIG. 20 with the administration assembly positioned proximal of the sealed package.

FIG. 22 is a cross-sectional side view of the delivery device of FIG. 21 with the administration assembly advanced partially distally to protrude a needle out of the housing.

FIG. 23 is a cross-sectional side view of the delivery device of FIG. 22 with the administration assembly advanced distally to dispense the contents of the sealed package through the needle.

FIG. 24 is a cross-sectional side view of the delivery device of FIG. 23 with the administration assembly advanced distally to permit retraction of the needle into the housing.

FIGS. 25 and 26 are top perspective views of a formed film of a sealed package in accordance with the present disclosure.

FIG. 27 is a side view of the formed film of FIGS. 25 and 26.

FIG. 28 is a side view of a sealed package including the formed film of FIGS. 25-27.

FIG. 29 is a partial cross-sectional side view of another example delivery device including the sealed package of FIGS. 25-28.

FIG. 30 is a partial cross-sectional side view of the delivery device of FIG. 29 within a compression assembly positioned distally and arrange to merge the first and second sealed compartments of the sealed package.

FIG. 31 is a partial cross-sectional side view of the delivery device of FIG. 30 with the first and second sealed compartments merged.

FIG. 32 is a partial cross-sectional side view of the delivery device of FIG. 31 with the compression assembly positioned proximal of the sealed package.

FIG. 33 is a partial cross-sectional side view of the delivery device of FIG. 32 with the compression assembly moved distally to dispense the contents of the sealed package from the delivery device.

FIG. 34 is a partial cross-sectional side view of the delivery device of FIG. 33 with the sealed package compressed by the compression assembly.

FIG. 35 is a partial cross-sectional side view of the delivery device of FIG. 34 with an actuator arm of the compression assembly detached from the compression panel of the compression assembly.

FIG. 36 is a partial cross-sectional side view of the delivery device of FIG. 35 with the actuator arm and actuator of the compression assembly advanced distally.

FIG. 37 is a partial cross-sectional side view of the delivery device of FIG. 36 with the sealed package retracted within the housing.

FIG. 38 is a top perspective view of another example delivery device in accordance with the present disclosure.

FIG. 39 is a top perspective view of a sealed package of the delivery device of FIG. 38.

FIG. 40 is a cross-sectional perspective view of the sealed package of FIG. 39.

FIG. 41 is a top perspective view of the sealed package of FIG. 39 and compression panel.

FIG. 42 is a top perspective view of the sealed package and a compression panel of FIG. 41 mounted to features of a compression assembly of the delivery device.

FIG. 43 is a cross-sectional perspective view of the assembly shown in FIG. 42.

FIG. 44 is a cross-sectional perspective view of the delivery device of FIG. 38.

FIG. 45 is a perspective view of the delivery device of FIG. 38 with the compression assembly advanced distally and contents of the sealed package dispensed from the sealed package.

FIG. 46 is a perspective view of the delivery device of FIG. 45 with the compression assembly and sealed package retracted into the housing of the delivery device.

FIG. 47 is a perspective view of the delivery device of FIG. 46 with the sealed package and compression assembly retracted within the housing.

FIG. 48 is a schematic cross-sectional side view of another example delivery device in accordance with the present disclosure.

FIG. 49 is a schematic cross-sectional side view of the delivery device of FIG. 48 with the sealed package and needle advanced distally within a housing of the delivery device.

FIG. 50 is a schematic cross-sectional side view of the delivery device of FIG. 49 with the compression member of the delivery device advanced further to merge compartments of the sealed package.

FIG. 51 is a schematic cross-sectional side view of the delivery device of FIG. 50 with the compression member advanced further distally to dispense contents of the sealed package through the needle.

FIG. 52 is a schematic cross-sectional side view of the delivery device of FIG. 51 with the compression member advanced further distally to retract the needle and sealed package within the housing.

In the following detailed description, reference is made to the accompanying drawings which form a part hereof, and in which is shown by way of illustrations specific embodiments for practicing the inventive aspect of the present disclosure. The leading digit(s) of the reference numbers in the figures usually correlate to the figure number, with the exception that identical or common components which appear in multiple figures may at times be identified by the same reference numbers. The embodiments illustrated by the figures are described in sufficient detail to enable those skilled in the art to practice the invention, and it is to be understood that other embodiments may be utilized and changes may be made without departing from the spirit and scope of the present disclosure.

### DETAILED DESCRIPTION

The present disclosure is directed to dispensers and packaging used for administering a therapeutic substance such as a vaccine to a patient. Typically, a sealed package is included having first and second sealed compartments that comprise two different substances such as, for example, a liquid and a powder. When the contents of the two compartments are mixed or merged together, the substance of the sealed packages is prepared for dispensing. The sealed package may comprise at least one frangible or rupturable seal used to separate the sealed compartments and/or temporarily block fluid communication between a dispenser or dispensing feature (e.g., a needle) and the contents of the sealed compartments prior to or after being merged. The term “dispenser” as used herein includes a structure configured to allow a substance to be expelled there through (e.g., from inside the compartment to exterior of the sealed package). Alternatively, one or more of the compartments may include an active ingredient dissolved on a meshed substance such as described in U.S. Published Patent Application Nos. 2009/0208585, 2008/0294100, and 2008/0026066, which are incorporated herein in their entirety by this reference.

Various constructions for sealed packages are disclosed herein. Further, the dispensers associated with the sealed packages may be constructed to both merge the sealed compartments together and dispense the contents of the sealed package. The sealed package typically includes a backing or substrate that defines a generally planar support surface. A formed film that defines the sealed compartments and frangible seals of the sealed package may be mounted to and extend or protrude from the support surface of the backing. The dispenser may apply a force to the sealed package that has a force component in a direction generally parallel with the support surface. This force may be applied to the sealed package to perform at least one of merging the first and second sealed compartments and dispensing the contents of the sealed package.

In some arrangements, the dispenser may be constructed to apply a force with a force component that is parallel to the support surface of the backing when both merging the first and second sealed compartments and when dispensing contents of the sealed package. The dispenser may also be constructed and operable to expose dispensing features such as, for example, a needle, needle fitment or Luer lock relative to a housing of the delivery device. In some embodiments the dispenser may be a cannula or a needle for invasive administration of the contents of the package to a tissue of a subject such as intradermal injection, subcutaneous injection, intramuscular injection or other injection methods known in the art for medical, cosmetic, veterinary, or other commercial dispensing applications. In other embodiments the dispenser is a dropper tip or a spray head for topical applications, oral applications, enteral applications, parenteral applications, ophthalmological applications, nasal, or ear treatment or medical, veterinarian, cosmetic, beauty or commercial applications known in the art. In yet other embodiments the dispenser is a surface applicator such as a brush, pad, or sponge for one of the applications described above. In yet other embodiments the dispenser comprises a connector or a fitting to connect to any of the above applicators or to connect to a tube or a bag to which the content of the package is to be dispensed.

Commercial applications include dispensing of glue, paint or dyes or samples of such, chemical agents for diagnostic or titration, glues, or other commercial applications know in the art. Those of skill in the art understand that dispensing assemblies may include plurality of applicators or replaceable applicators. Operating the dispenser to expose or retract the dispenser relative to the housing may occur concurrently with other operational steps such as merging the first and second sealed compartments and dispensing the contents of the sealed package.

The force component in the direction parallel with a support surface of the backing may be generally described as an axial or longitudinal force component. Typically, this axial or longitudinal force component is applied in a direction substantially perpendicular to a vertically applied force relative to the support surface of the backing. In some arrangements, the terms axial compression force, longitudinal compression force, and linear compression force or force component may be used to demonstrate or define application of the force component in the direction parallel with the support surface of the backing. The axial or longitudinal compression force may include force components in other directions such as in a lateral or side-to-side direction that may also be in a plane parallel with the support surface of the backing, or in a vertical direction that is perpendicular to both the axial or longitudinal direction and the lateral or side-to-side direction.

The delivery devices and dispensers disclosed herein may dispense the contents of the sealed package in any desired direction relative to the housing. In one example, the direction of dispensing contents of the sealed package may be arranged generally coaxial with the axially or linearly applied compression force. In other examples, the direction of dispensing may be generally perpendicular to the axially or linearly applied compression force.

One aspect of the present disclosure is directed to a dispenser wherein the dispenser (i.e., a needle) is exposed outside the housing of the dispenser only when the contents of the sealed package are being dispensed and not when the first and second sealed compartments of the sealed package are being merged together.

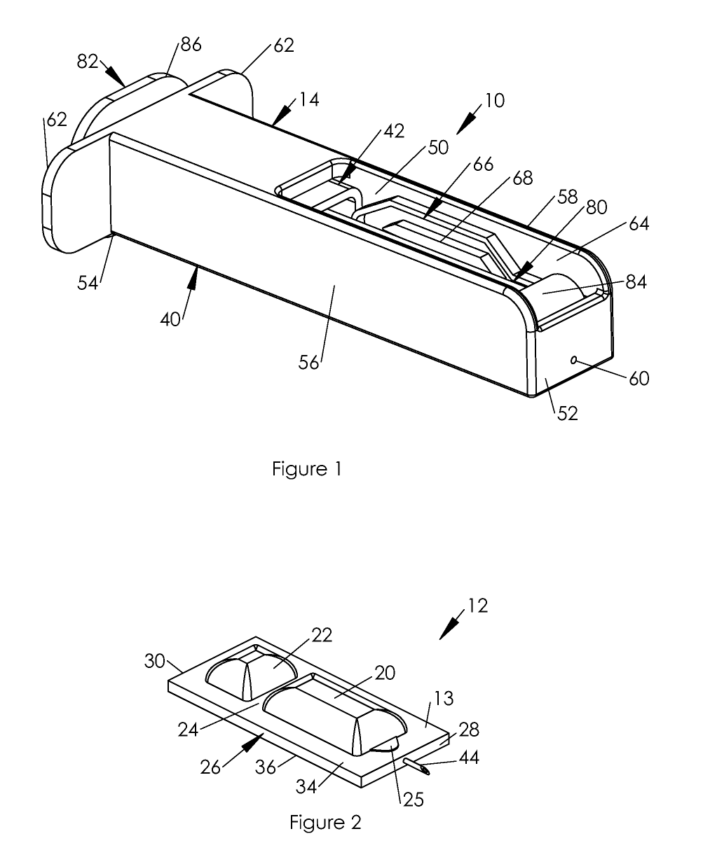

Referring now to FIGS. 1-6, an example delivery device 10 is shown and described. The delivery device 10 includes a sealed package 12 and an administration assembly 14 within which the sealed package resides. Referring to FIG. 2, the sealed package includes first and second sealed compartments 20, 22 and a frangible or rupturable seal 24 defined by formed film 13. The formed film 13 is mounted to a backing 26. The backing 26 includes opposed top and bottom surfaces 34, 36. The formed film 13 may be mounted to, for example, the top surface 34, such that the first and second sealed compartments 20, 22 protrude vertically upward from in a direction perpendicular relative to the top surface 34. The top surface 34 may be defined as a support surface. The top surface 34 may be generally planar. The backing 26 may include distal and proximal ends 28, 30. The backing 26 may have a longitudinal or axial dimension defined extending between the distal and proximal ends 28, 30.

Application of a compression force to either or both of the first and second sealed compartments 20, 22 typically ruptures the seal 24 to permit fluid flow between the first and second sealed compartments 20, 22. The sealed package 12 may include a second frangible or rupturable seal 25 interposed between the first and second sealed compartments 20, 22 and a delivery feature such as a needle 44. In one example, (e.g., see FIG. 2), the needle 44 is mounted directly to the sealed package 12. The sealed package 12 may include various features such as, for example, the second seal 25 that inhibits fluid flow through the needle 44 until after the first and second sealed compartments 20, 22 are merged together and the operator intentionally dispenses contents of the sealed package 12 through the needle 44.

Various dispensers are possible in place of or in addition to the needle 44. In some arrangements, the needle 44 or other dispenser may be moveable relative to the sealed package 12 to provide fluid communication with contents of the sealed package 12 only after the first and second sealed compartments 20, 22 are merged. Some example dispensers that include such moveable dispensing features are described in U.S. Published Application No. 2009/0171311, the disclosure of which is incorporated in its entirety by this reference.

A combined sealed compartment 32 may be formed after the seal 24 is ruptured (see FIG. 4). The combined sealed compartment 32 may include proximal and distal ends 38, 39.

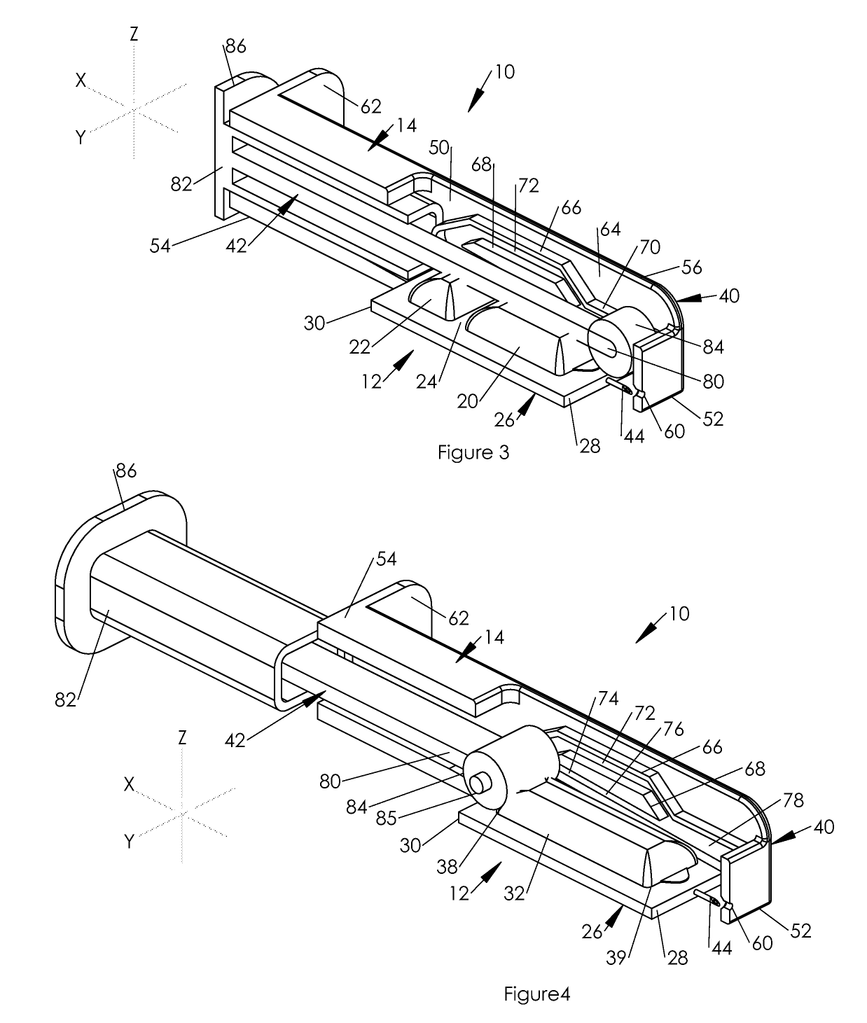

Referring now to FIGS. 3-6, the administration assembly may include a housing 40, a compression assembly 42 and a needle 44. As discussed above, the needle 44 may be integrated into the sealed package 12 or may be provided as a separate feature that is moveable relative to the sealed package 12.

The housing 40 includes a cavity 50, distal and proximal ends 52, 54, and first and second opposed side walls 56, 58. The distal end 52 may include a needle aperture 60. At least one finger tab or other grasping feature 62 may be mounted at the proximal end 54. First and second sidewalls 56, 58 may include an inner surface 64 to which at least one track 66 and ramp 68 are mounted. Relative positioning between the track 66 and ramp 68 define several positions for the compression assembly 42 that provide functions associated with the sealed package 12. For example, the track may define a combining section 70, an elevated section 72, a needle activate section 74, a compression or delivery section 76, and a needle retract section 78.

The compression assembly 42 may include distal and proximal ends 80, 82. Portions of the compression assembly 42 may be referred to as an actuator or an actuator portion (e.g., the proximal end 82). A compression member such as a roller 84 may be mounted at the distal end 80. The compression member may include a follower 85 that moves within the track 66 through the various sections 70, 72, 74, 76, 78 as will be described in further detail below. A thumb tab or other grasping member 86 may be positioned at the proximal end 82 and exposed outside of the housing 40 for easy access by an operator to actuate the compression assembly 42.

In operation, the administration assembly is arranged as shown in FIG. 3 with the roller 84 positioned distal of the first and second sealed compartments 20, 22. The follower 85 of the roller 84 resides in the needle retract section 78 of track 66 to permit retraction of the needle 44 within the housing 40. The operator then applies a force to the compression assembly 42 at the proximal end 82 in a proximal direction X relative to the housing 40. This application of force in the X direction may be referred to as linear actuation or actuation in a linear direction.

The follower 85 of the roller 84 moves through the combining section 70 of the track 66 and the roller 84 applies a compression force to the first sealed compartment 20. The compression force has a force component in the axial or linear direction, and may be referred to as an axial or linear compression force. Application of the compression force bursts the seal 24 to provide combining or merging of the first and second sealed compartments 20, 22. The first and second sealed compartments 20, 22 are merged into a combined sealed compartment 32 as shown in FIG. 4. The first sealed compartment 20 may be progressively collapsed as the roller 84 moves toward the second sealed compartment 22. An opening in the upper wall of the housing 40 allows for visual evaluation and confirmation of the mixture formation.

As the roller 84 moves from the rest position shown in FIG. 3 to the proximal position shown in FIG. 4, the follower 85 passes through an elevated section 72 that positions the roller 84 above the top of the combined sealed compartment 32 thereby relieving compression force otherwise applied to the combined sealed compartment 32.

The operator then applies another axial or linear directed force (i.e., in the X direction) in a distal direction to move the roller 84 into contact with the proximal end 38 of the combined sealed compartment 32 to apply another compression force. Application of this compression force moves the sealed package 12 distally within the housing 40 to advance the needle 44 through the needle aperture 60 to exposed position outside of the housing 40. The follower 85 of the roller 84 moves through a needle activation section 74 while advancing the needle 44. The combined sealed compartment 32 and the needle 44 may be caused to move by other arrangements such as the compression assembly 42 directly engaging with backing 26 and forcing the last to move.

The operator continues to apply a force in the distal direction X to progressively compress the combined sealed compartment 32. The compression assembly 42 may apply a compression force or have a force component that is generally axially or linearly directed in a plane parallel with the backing 26. The follower 85 may move through the compression or delivery section 76 of the track 66 while applying the compression force to the combined sealed compartment 32.

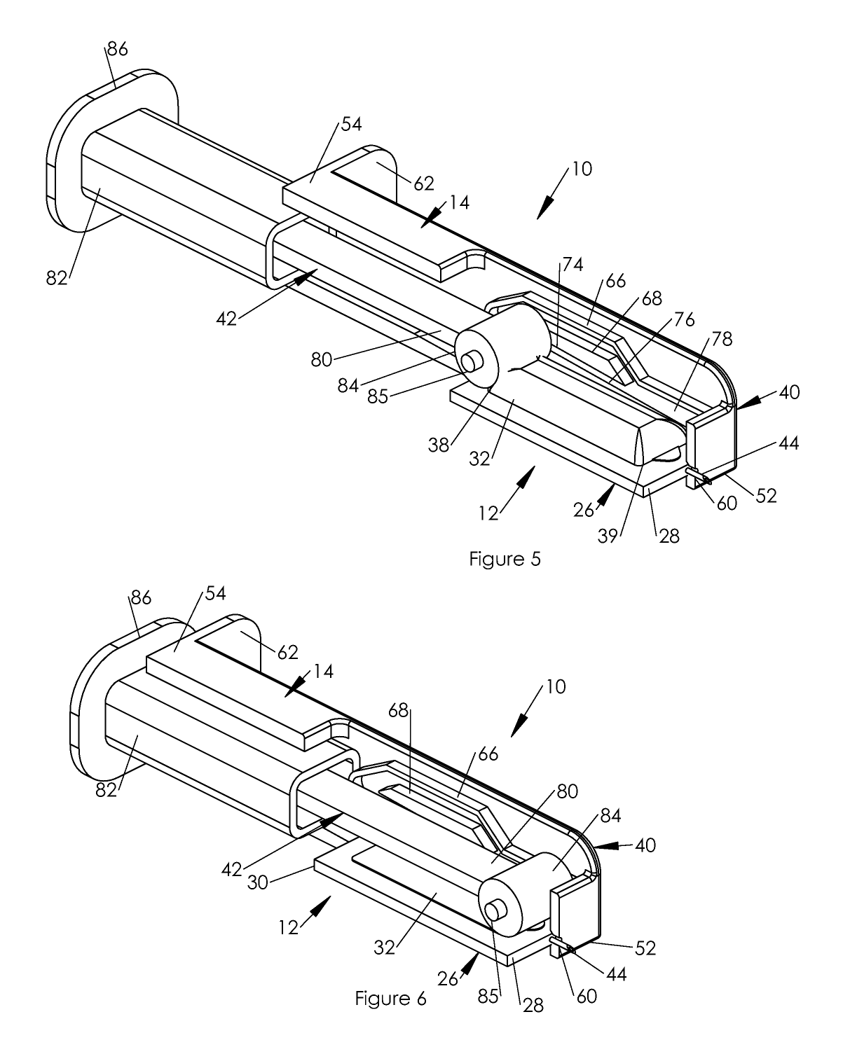

FIG. 6 illustrates the compression assembly 42 advanced distally until the roller 84 is positioned distally of the combined sealed compartment 32. The follower 85 enters a portion of the track 66 wherein the sealed package 12 is permitted to retract proximally to retract the needle 44 back into the housing 40. The follower 85 may be positioned in the needle retract section 78 in order for retraction of the needle 44 to occur. The needle retraction may be facilitated by a spring or another type of biasing member. The contents of the sealed package 12 may be completely or substantially dispensed from the delivery device 10 when the compression assembly 42 reaches the stage shown in FIG. 6.

The embodiment of FIGS. 1-6 provides for generally axial or longitudinal movement of the roller 84 by applying a generally axial or longitudinal force at the proximal end 82 via the thumb tab 86. Other arrangements are possible wherein force is applied in different directions (i.e., in a direction perpendicular to the direction of movement of the roller 84 within the housing 40) as will be described in further detail below.

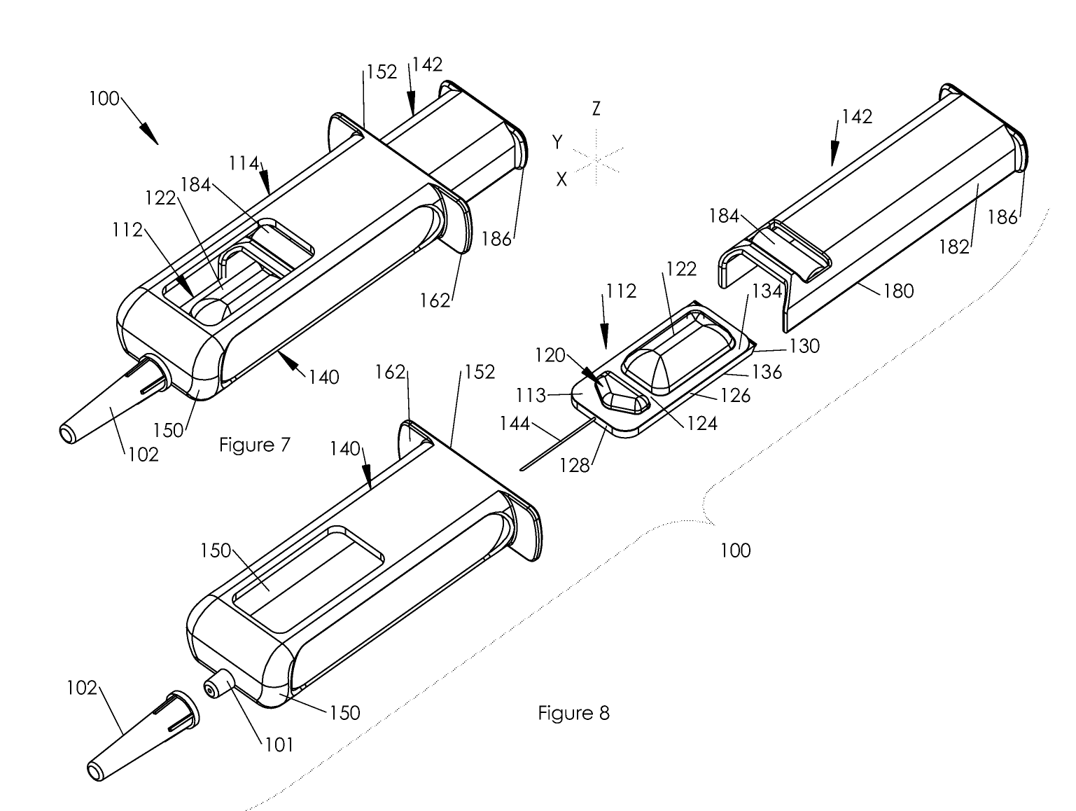

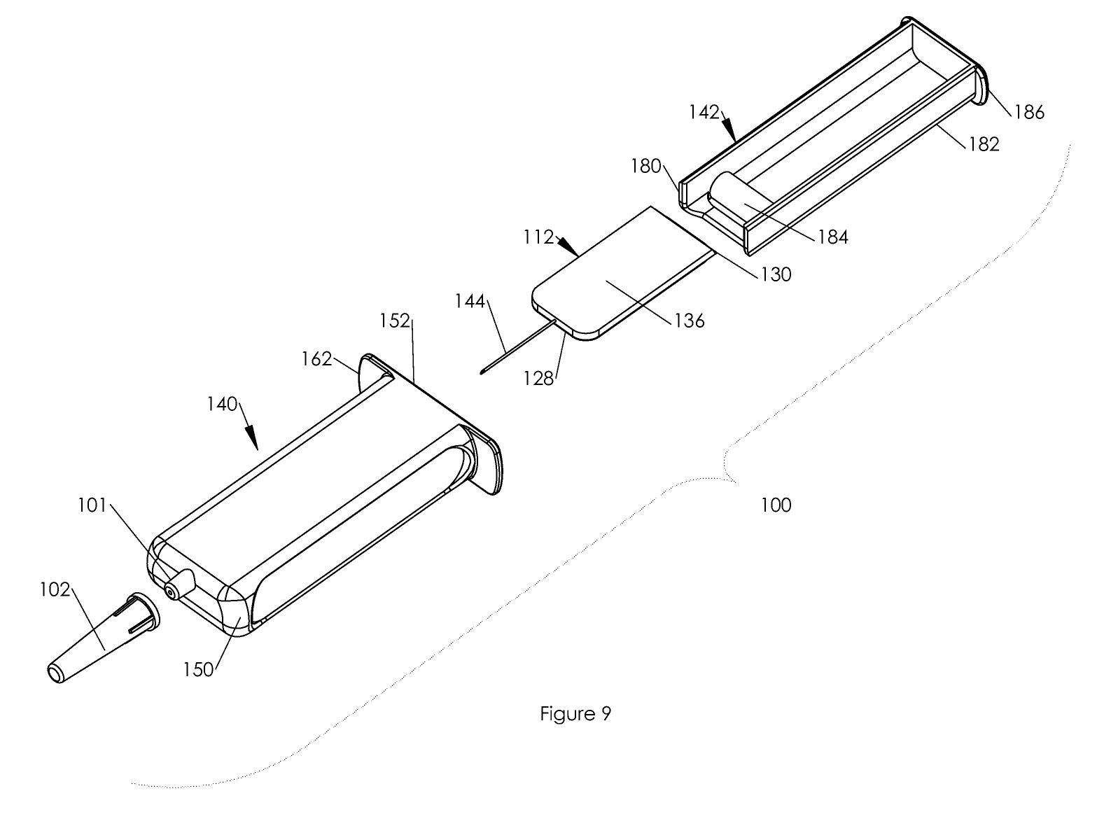

Referring now to FIGS. 7-9, another example delivery device 100 is shown and described. The delivery device 100 may include similar features as those described above relative to delivery device 10 and may further include a needle fitment 101 and needle cover 102. In at least one example, a sealed package 112 of the delivery device 100 may remain stationary within a housing 140 of an administration assembly 114. A needle 144 remains positioned outside of the housing 40 during operation of a delivery device 100. The needle cover 102 may protect and maintain concealment of the needle 144 until the delivery device 100 is used.

The sealed package 112 may include first and second sealed compartments 120, 122. At least one rupturable seal 124 of a formed film 113 is mounted to a backing 126. The backing 126 may include top and bottom surfaces 134, 136. The sealed package 112 includes distal and proximal ends 128, 130.

The housing 140 includes a cavity 150 and distal and proximal ends 152, 154. The compression assembly 142 includes distal and proximal ends 180, 182. A compression feature such as a roller 184 may be positioned at the distal end 180. A thumb tab 186 may be positioned at the proximal end 182 to help manipulate the compression assembly 142. Thumb tab 186 may be helpful in moving the compression assembly 142 relative to the housing 140 by contacting fingers of the operator against finger tab 162 of the housing 140 and a thumb against the thumb tab 186.

In operation, portions of the compression assembly 142 may be moveable in an axial or linear direction X to both merge the first and second sealed compartments 120, 122, and in a subsequent step dispense contents of the sealed package 112 through the needle 144. The roller 184 may initially be positioned proximal of the first and second sealed compartments 120, 122. Advancing the roller 184 may apply a compression force or force component to the second sealed compartment 122 thereby rupturing the seal 124 to merge the first and second sealed compartments 120, 122. Further advancing the roller 184 in the axial direction distally dispenses the contents of the merged first and second sealed compartments 122 through the needle 144.

An additional frangible or rupturable seal or other control feature may be interposed between the needle 144 and the merged first and second sealed compartments 120, 122. The additional seal may limit fluid communication with the needle 144 until after the first and second sealed compartments 120, 122 are merged and the operator intentionally applies a force to the compression assembly 142 to dispense the contents of the sealed package 112. The needle may be retractable in a similar arrangement to that disclosed in FIGS. 3-6.

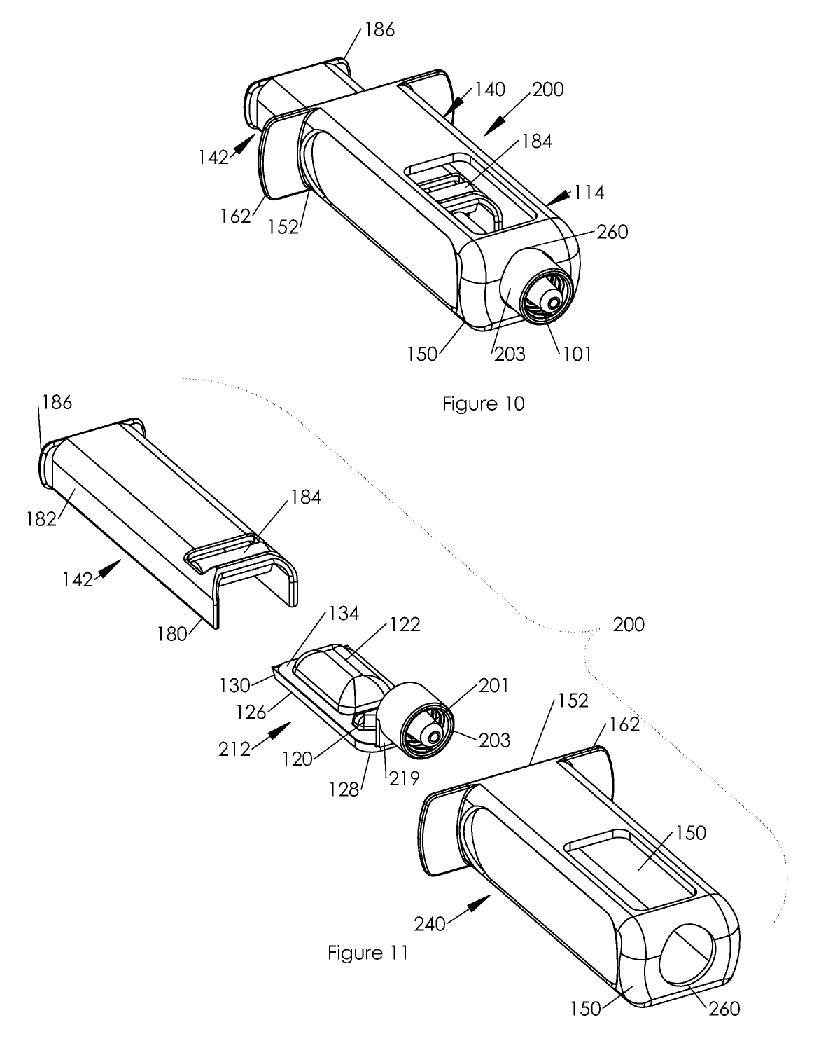

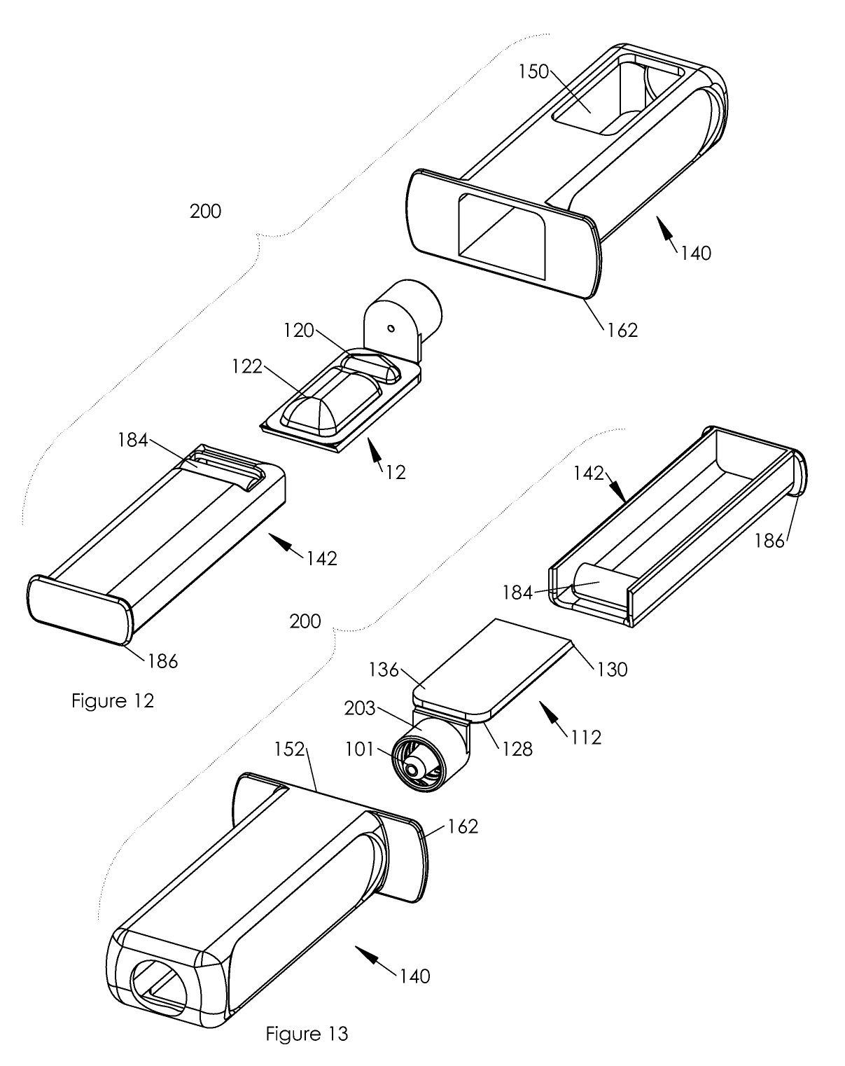

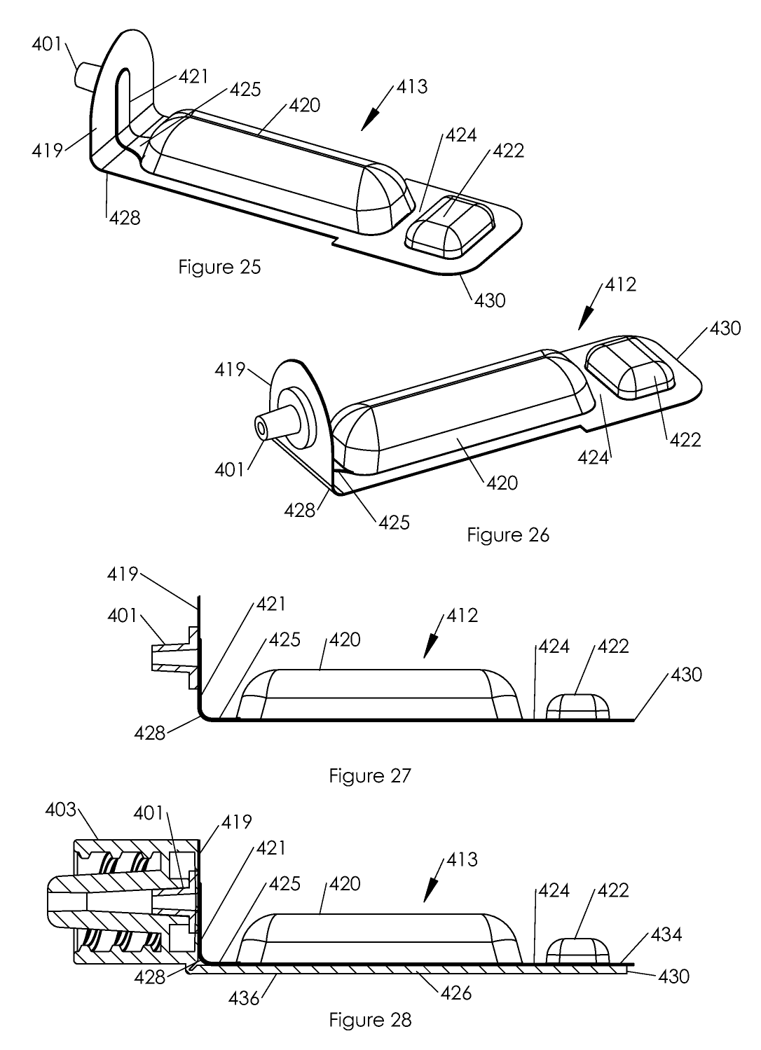

Referring now to FIGS. 10-13, another example delivery device 200 is shown and described. The delivery device 200 includes the same or similar construction for the sealing package 212 and administration assembly 114 of the delivery device 100. The delivery device 200 includes a housing 240 with a larger needle aperture 260 through which a Luer lock or hub 203 extends for mounting of a needle or other delivery structure. A needle fitment 101 may be positioned internal the Luer lock 203. The Luer lock 203 and needle fitment 101 may be mounted directly to the housing 240. Alternatively, the needle fitment 101 and Luer lock 203 may be mounted to the sealed package 212. The sealed package 212 shown in FIGS. 10-13 may have a similar construction and operation as the sealed package 412 described below with reference to FIGS. 25-37. In one example, the sealed package 212 may include multiple frangible or rupturable seals, wherein at least one of the frangible seals is defined between first and second sealed compartments 120, 122 and the needle fitment 101. A channel (not shown) may be also be defined in the sealed package 212 to provide such fluid communication between the sealed compartments 120, 122 and the needle fitment 101.

The needle fitment 101 and Luer lock 203 may be mounted to an extension 219 extending from the backing 126 of the sealed package 212. The extension 219 may be foldable such that the pouch is conveniently integrated with the backing and the extension. The extension 219 may be folded to bring the Luer lock connector into a desired orientation. The sealed package 212 may be used as a reservoir or a cartridge for various applications. In a similar manner, sealed packages 12, 112 of the other embodiments disclosed herein may be used as a reservoir or cartridge.

The Luer lock 203 may be constructed for mounting a standard dispenser such as a needle. The Luer lock 203 may include a plurality of threads to help retain the needle. Other mounting structures may be used in place of the Luer lock 203 of delivery device 200 and the needle fitment 101 of the delivery device 100 described above. Further, other dispenser structures besides a needle may be used with or without the Luer lock 203 for dispensing including, for example, a mini needle, a micro-needle, a connector, a male Luer connector, a female Luer connector, a topical applicator, a spray nozzle, an ophthalmic applicator, an ear dispenser, a fitting, and a tube.

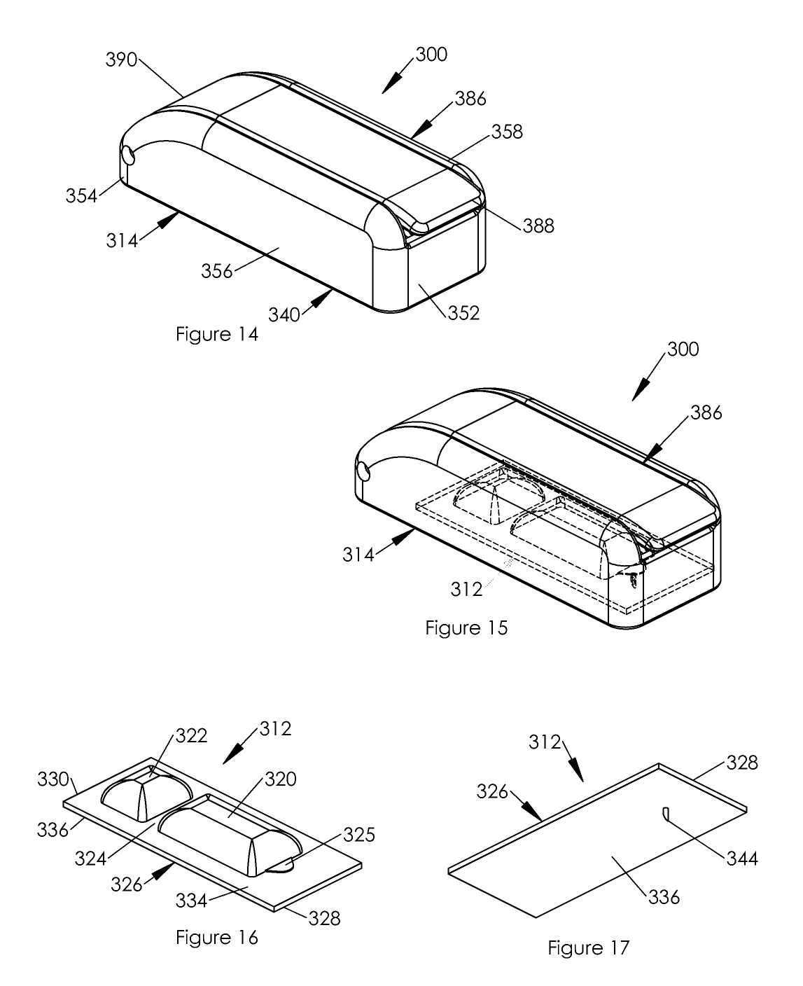

Referring now to FIGS. 14-24, a further example of delivery device 300 is shown and described. A delivery device 300 includes a sealed package 312 and an administration assembly 314. The administration assembly 314 includes a hinged handle 386 that moves a compression member within a housing 340. Moving the handle 386 between various rotated positions provides merging of sealed compartments of the sealed package 312, and in a subsequent step dispenses the contents of the sealed package from the delivery device 300. Further, the delivery device 300 may provide for dispensing in a different direction than the direction of movement of the compression member within the housing. For example, the delivery device 300 may include a needle that extends from a bottom side thereof. In use, the delivery device 300 may rest upon the skin surface of a patient with a bottom side of the housing 340 in contact with the patient. Rotating the handle 386 merges the sealed compartments of the sealed package 312, causes a needle to penetrate the patient's skin, and dispenses the contents of the sealed package 312 into the patient via the needle.

The sealed package 312 is shown in further detail in FIGS. 16 and 17. The sealed package includes first and second sealed compartments 320, 322 and at least one rupturable seal 324. An additional rupturable seal may be interposed between the first and second sealed compartments 320, 322 and a needle 344 (see FIG. 17). The additional rupturable seal 325 may be positioned along the backing 326 such as, for example, at a distal end of the first sealed compartment 320. The additional rupturable seal 325 may inhibit fluid flow between the sealed compartments 320, 322 and the needle 344 until the operator intends to dispense the contents of the sealed package 312.

The backing 326 may include top and bottom surfaces 334, 336. The sealed package 312 may include distal and proximal 328, 330. The seal 324 is ruptured upon applying a compression force to either one of the first and second sealed compartments 320, 322 to create merging of the first and second sealed compartments 320, 322 and form a combined sealed compartment 332 (see FIG. 20). The combined sealed compartment 332 combines proximal distal and distal ends 338, 339.

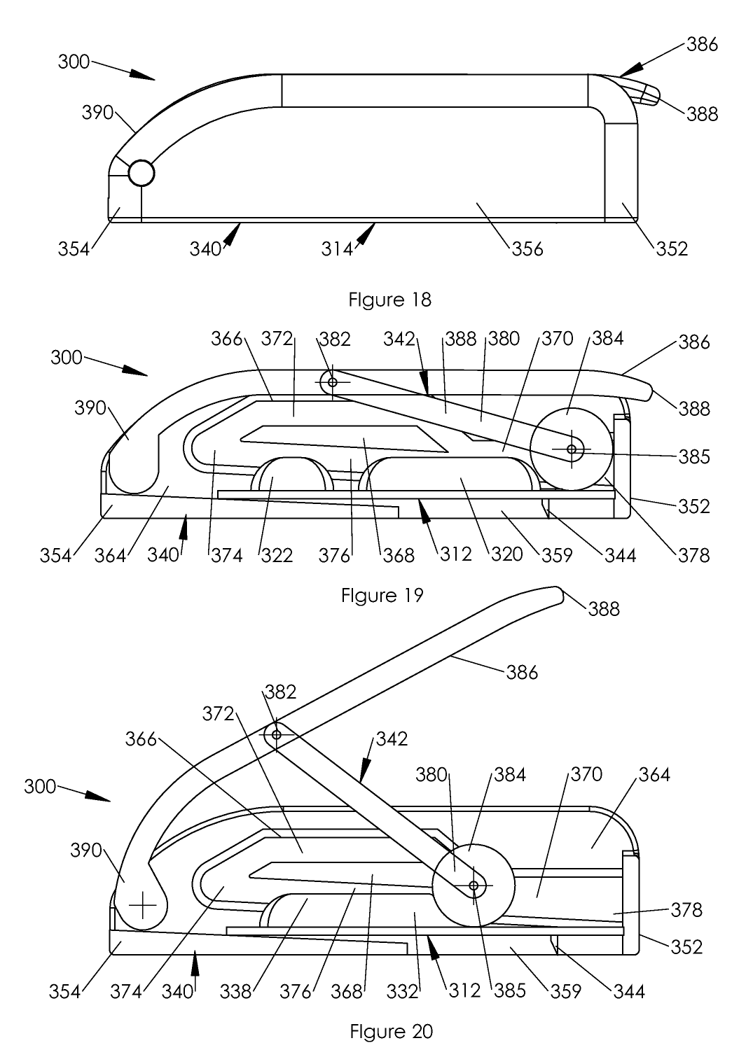

Referring now to FIGS. 18-24, the administration assembly 314 includes a housing 340, a compression assembly 342 and a needle 344. The housing 340 includes a cavity 350, distal and proximal ends 352, 354, and first and second sidewalls 356, 358. The sidewalls 356, 358 define inner surfaces 364 upon which a track 366 and ramp 368 are mounted. The track 366 and ramp 368 define various sections 370, 372, 374, 376, 378 to help determine a position of compression features such as roller 384 relative to the sealed package 312. The housing 340 also includes a bottom opening 359 that permits movement of the needle 344 into and out of the housing 340.

The compression assembly 342 includes distal and proximal ends 380, 382, a roller 384 having a follower 385, and a handle 386 having a free end 388 and a connection end 390. The connection end 390 is mounted to the housing 340 at the proximal end 354.

The roller 384 is connected to an actuator in the form of a handle 386 with a connection arm 398. Raising and lowering the handle 386 moves the roller 384 within the housing 340. A follower 385 extending from the roller 384 moves within the track 366 through various track sections as noted above.

In operation, the handle 386 typically begins in a closed position or state as shown in FIGS. 18 and 19. In this closed state, the roller 384 is arranged to permit the sealed package 312 and associated needle 344 to remain positioned within the housing 340. The follower 385 rests within a needle retracted position 378 (also referred to as a rest state).

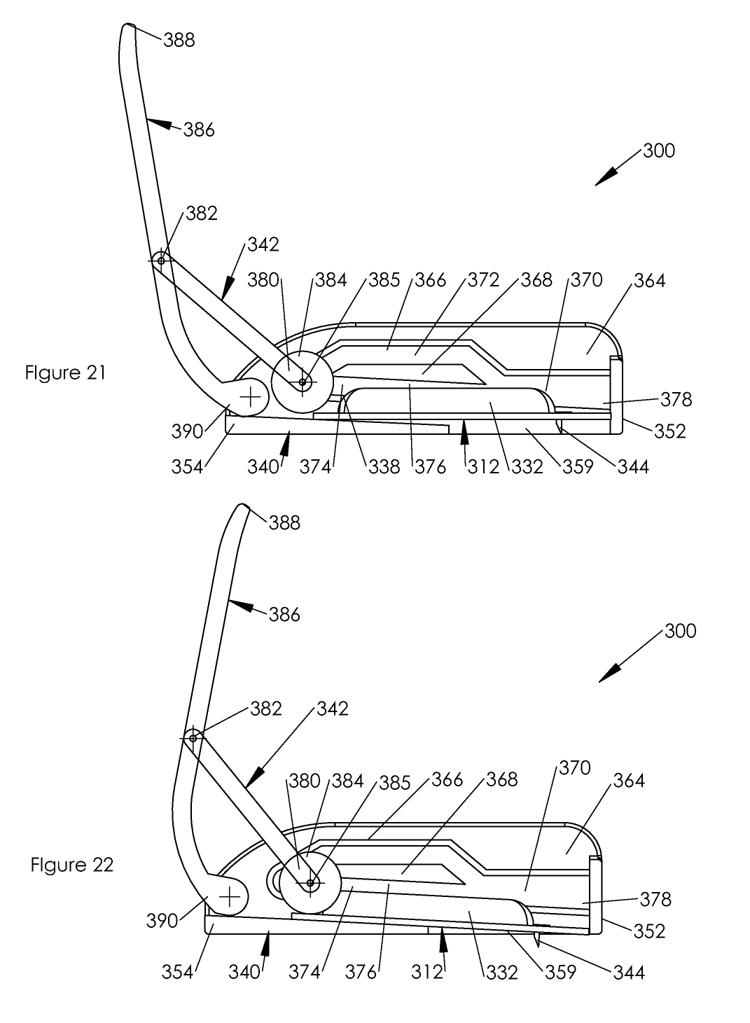

The handle 386 is pivoted about the pivoted connection to the housing 340 at the proximal end 354. The follower 385 moves through a combining section 370 of the track wherein the roller 384 applies a compression force to the first sealed compartment 320. Application of the compression force ruptures the seal 324 and merges the sealed compartments 320, 322. The follower 385 then passes into an elevated section 372 wherein the roller 384 moves out of contact with the combined sealed compartment 332 into a position proximal of the combined sealed compartment 332 as shown in FIG. 21.

Moving the handle 386 back toward the closed position advances the roller 384 into a needle activation section 374 of the track wherein the roller 384 contacts a proximal end 338 of the combined sealed compartment 332. Application of the compression force to the combined sealed compartment 332 with the roller 384 rotates the sealed package 312 to advance the needle 344 out of the housing 340 as shown in FIG. 22. The sealed package 312 may be advanced by other arrangements such as engagement of the connecting arm 398 with the backing 326. Further rotation of the handle 386 towards the closed position applies further compression forces to the combined sealed compartment 332 to dispense the contents of the sealed package 312 through the needle 344. In some arrangements, the second or additional rupturable seal 325 may rupture upon application of the additional compression force to permit fluid communication between the needle 344 and the combined sealed compartment 332.

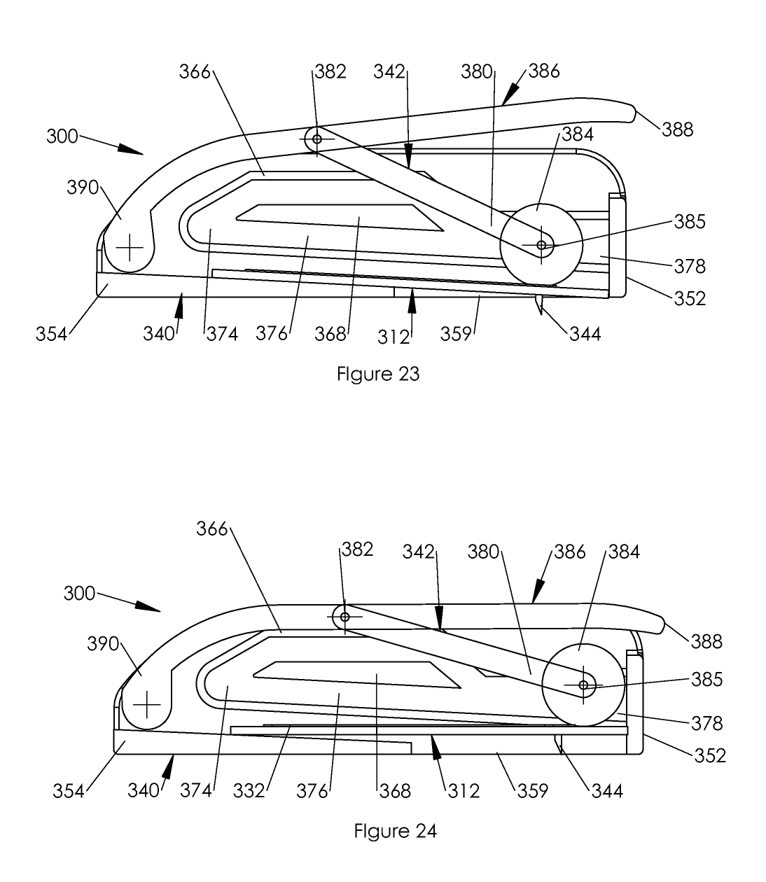

The follower 385 moves through a compressed or delivery section 376 wherein the roller 384 moves distally to dispense the contents of the sealed package 312. The handle 386 is movable towards the closed position as shown in FIG. 23 until the roller 384 passes to a location distal of the combined sealed compartment 332.

Further closing the handle 386 towards the closed state shown in FIG. 24 moves the follower 385 into the needle retract section of 378 of the track 366 that permits rotation of the sealed package 312 back into the housing to retract the needle 344 as shown in FIG. 24. The retraction of the sealed package 312 may be facilitated by a spring or another biasing member.

Other track instructions, followers and compression features may be used in combination with or in place or those features shown with reference to FIGS. 14-24 and the embodiment of FIGS. 1-6 to provide merging of sealed compartments of the sealed package, dispensing of the contents of the sealed package, and movement of a needle relative to the housing.

Referring now to FIGS. 25-37, an alternative sealed package 412 and associated delivery device 400 is shown and described. Referring first to FIGS. 25-27, a formed film 413 for use with a sealed package 412 (see FIG. 28) includes first and second sealed compartments 420, 422 and first and second frangible seals 424, 425. The formed film 413 may include a primary portion within which the first and second sealed compartments 420, 422 are formed, and an extension 419 arranged generally perpendicular to the primary portion. A needle fitment 401 may be mounted to the extension 419. A channel 421 may be defined in the formed film 413 between the first sealed compartment 420 and the needle fitment 401. The second seal 425 may be interposed between the channel 421 and the first sealed compartment 420. The first seal 424 may be interposed between the first and second sealed compartments 420, 422.

The formed film 413 may be mounted to a backing 426 that includes top and bottom surfaces 434, 436. A hub or Luer lock 403 may be mounted to the needle fitment 401. The hub 403 may be integrally formed with backing 426. Alternatively, the hub 403 may be formed as a separate piece from the backing 426 and separately mounted to the formed film 413. The sealed package 412 may include distal and proximal ends 428, 430. Application of a compressive force to either one of the first and second sealed compartments 420, 422 may result in formation of a combined sealed component 432 having proximal and distal ends 438, 439 (see FIG. 31). The first and second seals 424, 425 may be configured such that the first seal 424 has a weaker pilling force so that it will rupture first to permit merging of the first and second sealed compartments 420, 422 prior to dispensing the contents of the sealed package 412. Alternatively, the first and second seals 424, 425 may have the same pilling strength. Applying a compressive force in a particular direction to either one of the first and second sealed compartments 420, 422 may be useful in determining which of the first and second seals 424, 425 ruptures first. In the example of FIGS. 25-37, a compression force applied in a proximal direction to the first sealed compartment 420 may cause the first seal 424 to rupture. Later application of an compression force to the combined sealed compartment 432 in a distal direction (see FIG. 33) may cause rupturing of the second seal 425 after rupturing of the first seal 424 even if the second seal has the same or lower pilling strength.

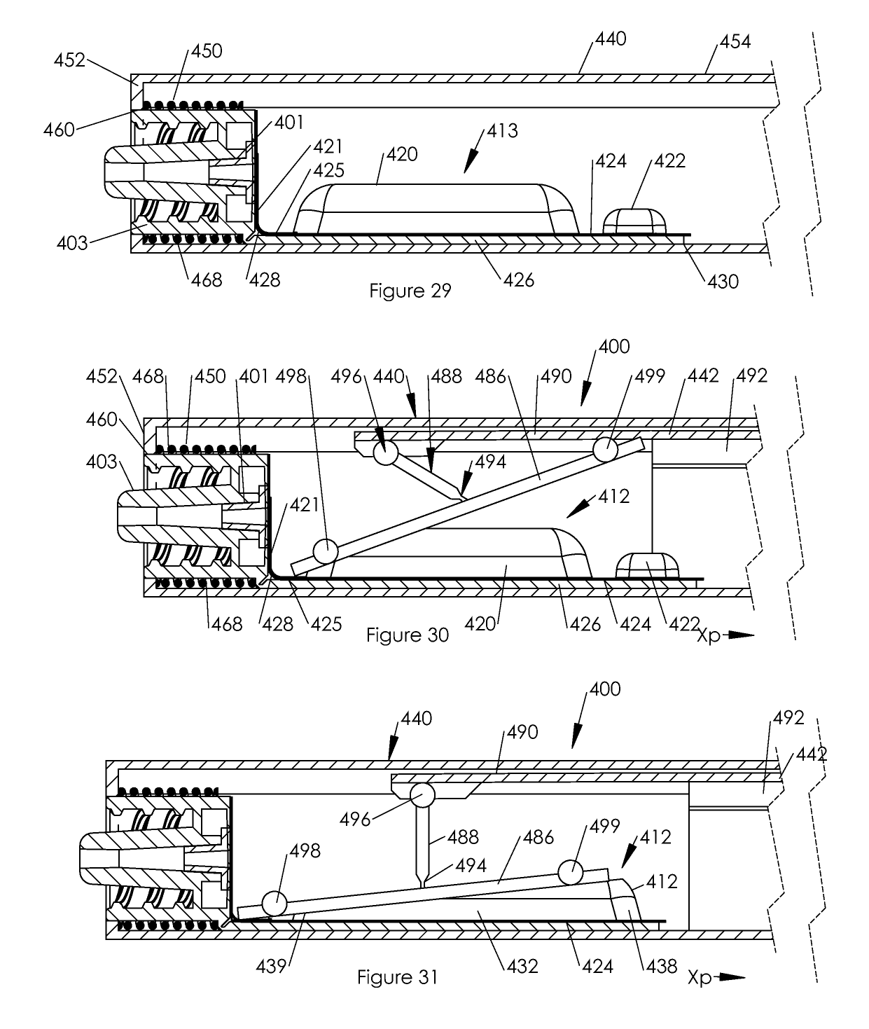

Referring now to FIGS. 29-37, a delivery device 400 includes the sealed package 412 and an administration assembly 414. The administration assembly 414 includes a housing 440 and a compression assembly 442. The housing 440 includes a cavity 450, distal and proximal ends 452, 454, and an aperture 460 defined at the distal end 452. A biasing member 468 may be positioned in the housing 440 interposed between the sealed package 412 and a distal end surface of the housing 440. Biasing member 468 may bias the sealed package 412 to a position within the housing 440 with the hub 403 retracted within the housing 440 as shown in FIG. 37.

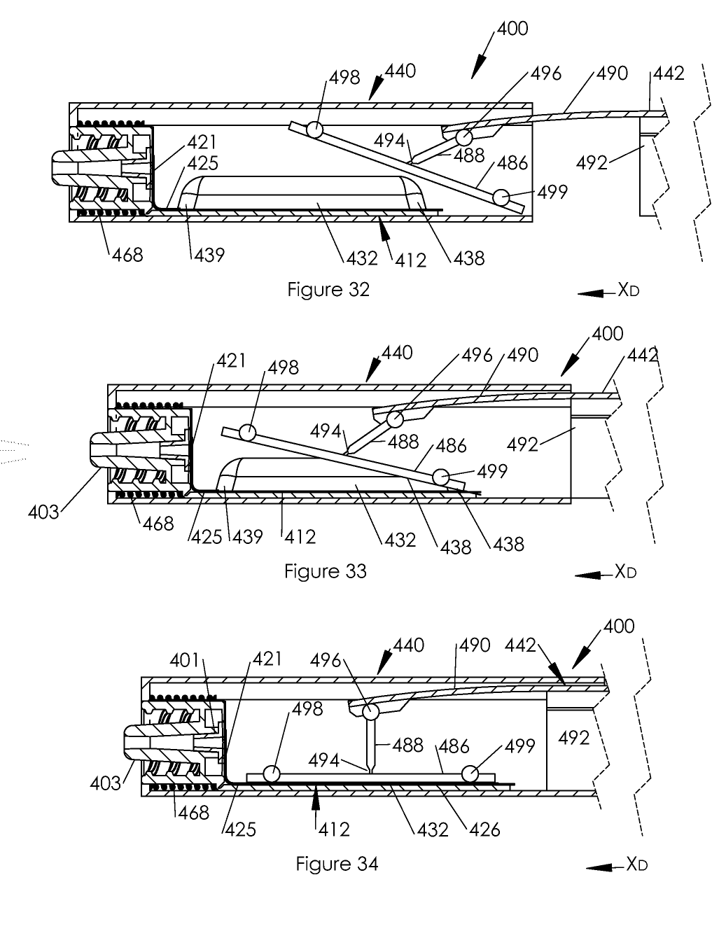

The compression assembly 442 includes a compression panel 486, a connection arm 488, an actuator arm 490 and an actuator 492. The connection arm 488 is connected to the compression panel 486 with a hinge such as a living hinge structure. A pivot joint 496 may be provided at an interface between the connection arm 488 and the actuator arm 490. The actuator arm 490 may comprise a generally flexible structure that permits some bending as shown in at least FIGS. 32-34. The compression panel 486 may include first and second guiding members or followers 498, 499. The followers 498, 499 may interface with features along the length of the housing 440 such as, for example, tracks, grooves, ramps, or other features (not shown) that force the compression panel 486 into various orientations within the housing 440 during axial movement of the compression assembly 442 relative to the housing 440.

Referring now to FIG. 30, in an initial operation state, the compression assembly 442 is positioned within the housing 440 with the compression panel 486 arranged at a distal end of the first sealed compartment 420. Axial movement of the compression assembly 442 in the proximal direction (direction X_{P}) applies an compression force to the first sealed compartment 420 that ruptures the first seal 424 to merge the first and second sealed compartments 420, 422 (see FIG. 31). Further axial movement of the compression assembly 442 in the proximal direction moves the compression panel 486 out of contact with the combined sealed compartment 432 and to a position at a proximal end 428 of the combined sealed compartment 432 as shown in FIG. 32.

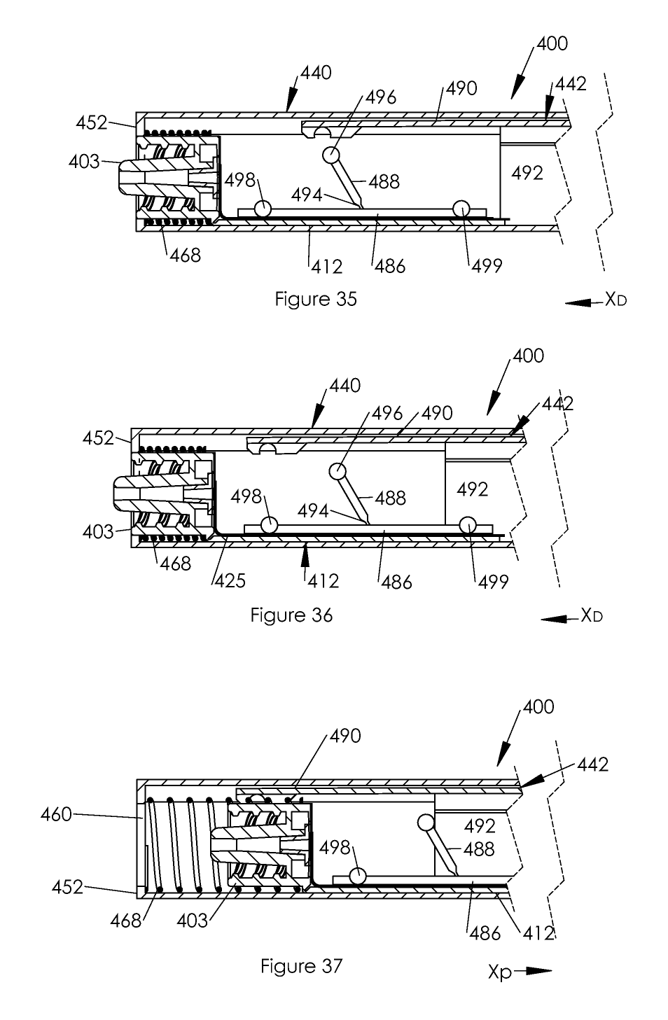

Axial movement of the compression assembly 442 in the distal direction (X_{D}) applies a compression force to the combined sealed compartment 432 as shown in FIG. 33 to create dispensing of the contents of the sealed package 412 by rupturing the second seal 425 and passing the contents through the channel 421 and out of the hub 403. The compression assembly 442 is advanced distally until the combined sealed compartment 432 is compressed as shown in FIG. 34. Further advancing the compression assembly 442 in the distal direction disconnects the actuator arm 490 from the connection arm 488 as shown in FIG. 35. This disconnection permits the sealed package 412 to begin to move axially in a proximal direction as shown in FIG. 36. With the sealed package 412 permitted to move proximally within the housing 444, the biasing force applied by the biasing member 468 moves the hub 403 internal the housing 444 and inaccessible by the operator. The sealed package 412 may be held at the distal position by, for example, a detent mechanism that allows the sealed compartment to retract when the actuator has substantially completed its travel to the distal position.

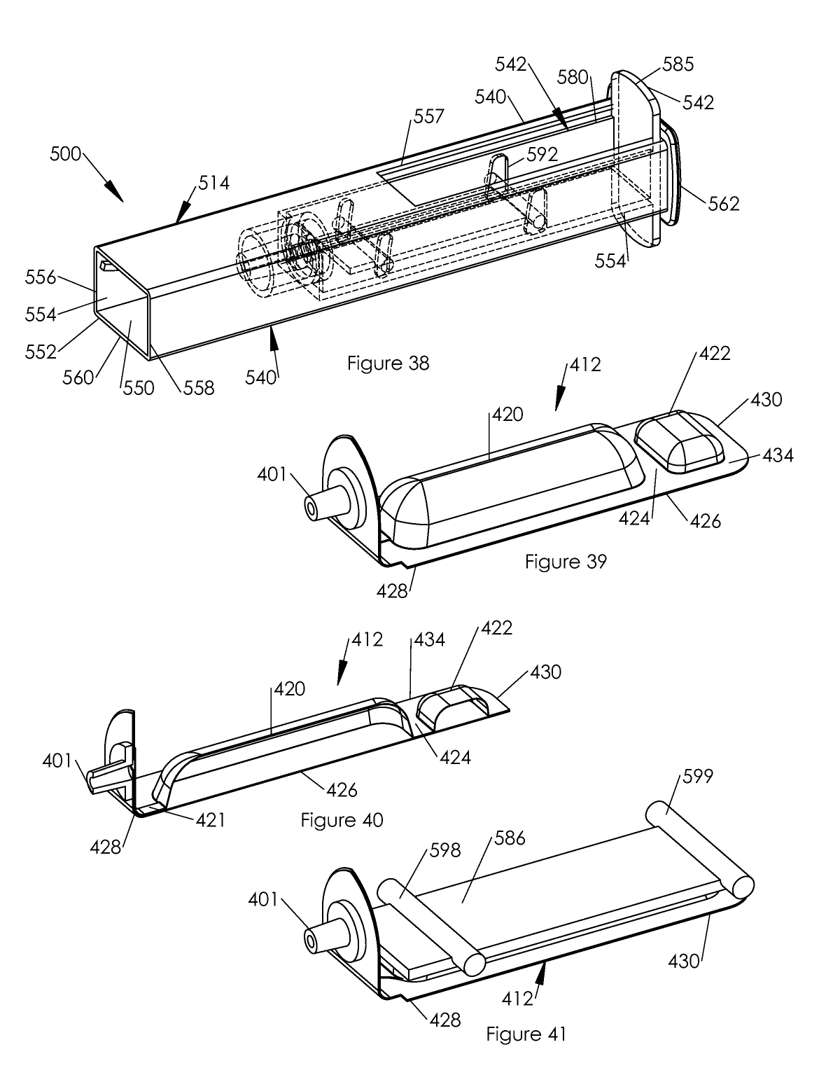

Referring now to FIGS. 38-48, an example delivery device 500 is shown and described including a sealed package 412 and an administration assembly 514. The sealed package 412 may have the same or similar features and functions as described above with reference to FIGS. 25-37.

The administration assembly 514 includes a housing 540 and a compression assembly 542. The housing 540 includes a cavity 550, distal and proximal ends 552, 554, first and second sidewalls 556, 558 each having an inner surface 564, and a plurality of sets of track members 566, 567. An aperture 560 is defined at the distal end 552 to permit a portion of the delivery device 500 (e.g., the hub 403) to extend out of the housing 540. The housing 540 may also include top and bottom openings 557, 559 through which portions of the compression assembly 542 extend.

The compression assembly 542 includes distal and proximal ends 580, 582 and a thumb tab 585 extending from the proximal end 582. The compression assembly 542 also includes a compression panel 586 and first and second sets of slots 590, 592 through which followers 598, 599 of the compression panel 586 protrude. The followers 598, 599 contact the sets of track members 566, 567 to move the compression panel 586 relative to the sealed package 412 as the compression assembly 542 moves relative to the housing 540.

The needle fitment 401 may comprise a flange having a relatively small footprint or profile. One advantage of providing a smaller foot print for the needle fitment 401 is that it can be attached to a wall of the sealed package without extending the overall size of the package. The following method steps may be followed to provide such attachment to a wall of the sealed package:
The fitment is attached to the outer wall of the sealed package 412 (e.g., by heat welding).
The first wall and the second wall of the sealed package 412 are attached, for example, by heat welding, around the compartments 420, 422 and around the channel 421 leading to the fitment. Due to the small footprint of the fitment, the first wall and the second wall may be sealed together around the needle fitment 401 to complete a sealed channel without extending the size of the sealed package.
A Luer lock connector is attached to the fitment. If the Luer lock is attached directly to the sealed package, the sealed package may have to be extended to laterally exceed the size of the Luer lock to allow for sealing between the first wall and the second wall of the sealed package.

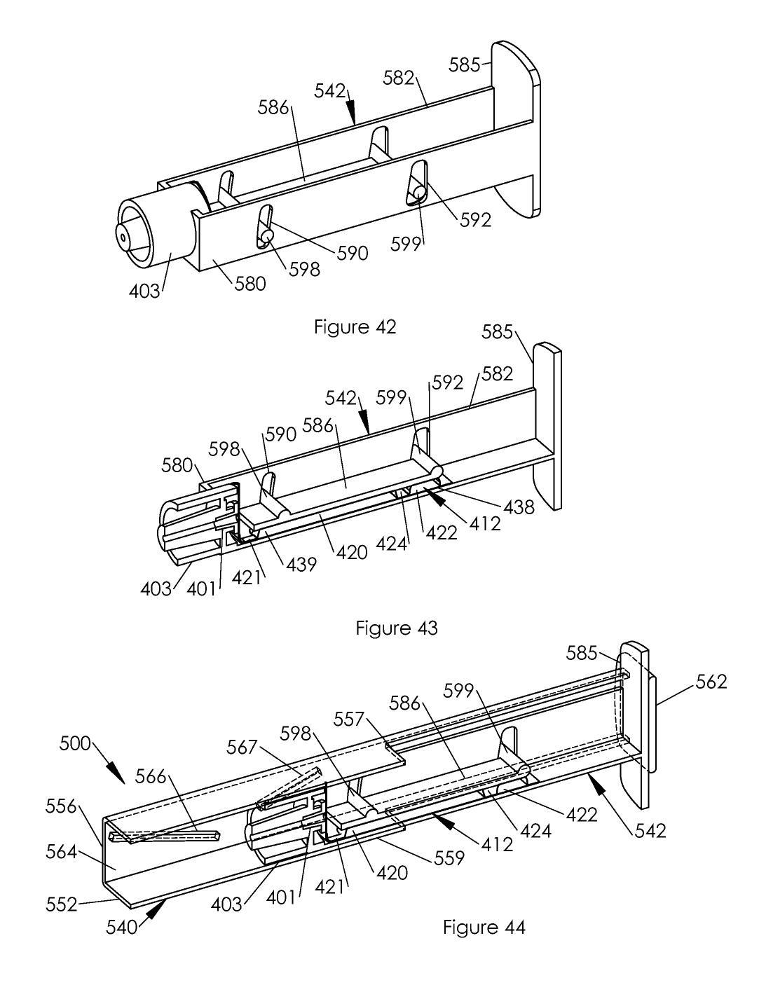

Referring to FIG. 41, the compression panel 586 is sized and configured to be positioned vertically above the sealed package 412 within the housing 540. As the followers 598, 599 contact the track members 566, 567, the compression panel 586 applies compression forces to the first and second sealed compartments 420, 422 and the combined sealed compartment 432 in proximal or distal directions in order to merge the first and second sealed compartments 420, 422 and dispense the contents of the sealed package 412. FIGS. 42-44 illustrate the sealed package 412 and administration assembly 514 in a rest position prior to actuation of the delivery device 500.

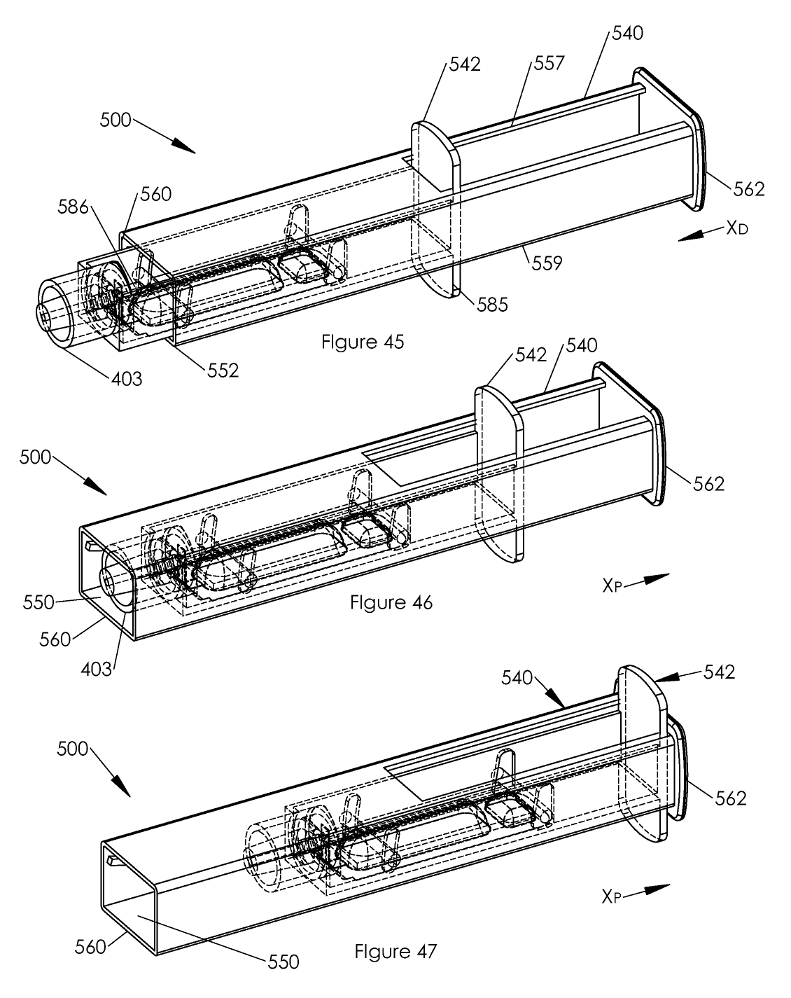

Referring to FIG. 45, in at least one operational step of the delivery device 500, the compression assembly 542 is advanced distally in the direction X_{D} to dispense the contents of the sealed package 412. The thumb tabs 585 of the compression assembly 542 may move within the openings 557, 559 to control the amount of axial movement of the compression assembly 542 relative to the housing 540. Retracting the compression assembly 542 relative to the housing 540 in the direction X_{P} moves the hub 403 into a position inaccessible after dispensing the contents of the sealed package 412. In some arrangements, the hub 403 may be retracted within the housing 540 a distance sufficient to conceal within the housing a needle or other dispenser that is attached to the hub 403.

Referring now to FIGS. 49-52, a delivery device 600 includes a sealed package 612 and an administration assembly 614. The sealed package 612 includes first and second sealed compartments 620, 622 and at least a first frangible seal 624. In some embodiments, one or more additional rupturable seals are included for example, to control fluid flow between a needle 644 and at least one of the first and second sealed compartments 620, 622.

The sealed package 612 may also include a backing 626 having a top surface 634 to which the first and second sealed compartments 620, 622 are mounted. The sealed package 612 may include distal and proximal ends 628, 630. Upon application of a compressive force to at least one of the first and second sealed compartments 620, 622, a combined sealed compartment 632 may be formed upon rupturing of the seal 624. In a combined sealed compartment 632 may include proximal and distal ends 638, 639 as shown in FIG. 50.

The administration assembly may include a housing 640 and a compression assembly 642. The housing 640 may include a cavity 650, distal and proximal ends 652, 654 and a needle aperture 660 defined at the distal end 652. The compression assembly 642 may include first and second ends 680, 682, a curved compression portion 684, and an actuator 686.

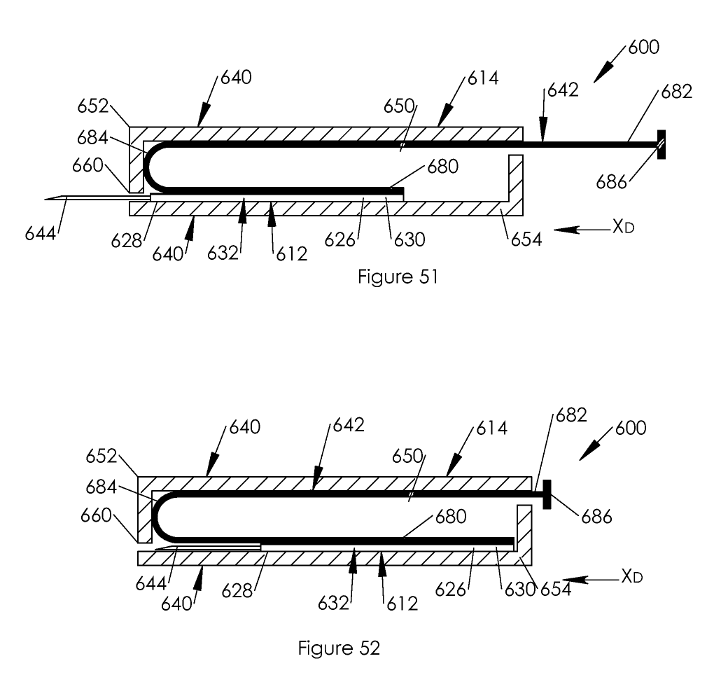

In operation, the sealed package 612 is positioned within the housing 640. The compression assembly 642 is positioned proximal of the sealed 612 and arranged to apply a compression force to the second sealed compartment 622. Applying a force in the direction X_{D} at the actuator 686 advances the sealed package 612 to extend a needle 644 out of the needle aperture 660 of the housing 640 as shown in FIG. 49. Further distal movement of the actuator 686 applies a compression force to the second sealed compartment 622 that ruptures the seal 624 to create a combined sealed compartment 632 as shown in FIG. 50. Still further distal advancement of the actuators 686 applies additional compression force at a proximal end 638 of the combined sealed compartment 632 to dispense the contents of the sealed package 612 through the needle 644. Still further distal advancement of the actuator 686 retracts the needle 644 and sealed package 612 back into the housing 640 as shown in FIGS. 51 and 52.

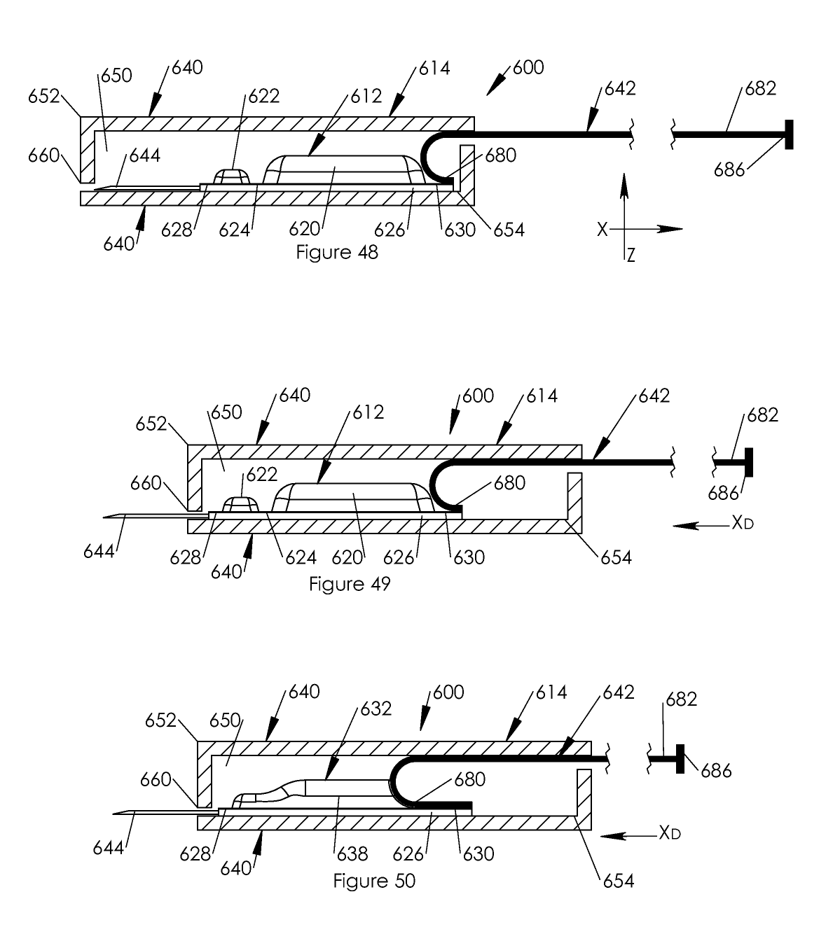

The compression assembly 642 may be constructed as a ribbon or band type device having a greater width than thickness. In some arrangements, the compression assembly 642 may include portions along its length that are relatively rigid and other portions along its length that are relatively flexible that promote the bending shown in FIGS. 48-52. Delivery device 600 may provide both merging of the first and second sealed compartments 620, 622 and dispensing the contents of the sealed package 612 by actuating the compression assembly 642 in the distal direction only.

The compression assembly 642 accepts a shape of the lower wall 654 to more efficiently extract the volume of the merged sealed compartments 620, 622. Going back to the arrangements of FIGS. 30-37 and FIGS. 42-47, the compression panel and the backing may be made such that at least one of the surfaces of the compression panel and backing is different in shape than the opposite surface. During compression of the compartment, at least one of the compression panel and backing substantially accepts the shape of the other, thereby extending an exerted force on the compartment and improving extraction of the content of the compartment. The surfaces of the compression panel and backing that interface may each have a contoured shape.

Although the example sealed packages illustrated in the attached figures and described herein include two separate distinct sealed compartments, other configurations are possible for use with the various delivery devices disclosed herein. For example, the sealed package may include a single sealed compartment that does not require merging with another sealed compartment. Another example, three or more sealed compartments are supported on a single backing and are merged together to form a single combined sealed compartment during operation of the delivery device. In another example, several compartments are arranged in parallel on the same backing and are operated or acted upon by the same compression panel. Furthermore, while a single needle or other dispenser is included in the examples disclosed herein, other arrangements may include multiple dispensers such as multiple needles. In some arrangements, a separate needle may be associated with separate sealed compartments of the sealed package.

Referring to the various sealed package embodiments disclosed herein, improvements in manufacturing and assembly of the sealed packages are possible by altering a physical state of the contents of each of the sealed compartments. In at least one example, one of the sealed compartments includes a liquid such as water. The liquid is frozen or partially frozen prior to positioning in the compartment. In one example, water is frozen into aliquot portions for improved ease of handling. In another example, at least one of the sealed compartments includes a powder substance such as a powder vaccine. The powder may be slightly compressed into a pellet shape or into a plurality of small conglomerates of powder material. Such compressed or slightly solidified portions of powder material may be easier to handle than powder during the process of filling the sealed compartments. The powder may be slightly compressed so that the powder may be handled like a solid during the process of filling the sealed package, but not compressed enough to significantly reduce the dissolution rate after reconstitution with a liquid. The powder may be separated again into loose powder after sealing the sealed compartments of the sealed package by external mechanical manipulations prior to assembling the sealed package with a delivery device.

An example method of administering a therapeutic substance in accordance with the present disclosure includes providing a dispensing device having an administration assembly and a sealed package. The method includes applying a linear force to at least one sealed portion of the sealed package to activate the therapeutic substance. The method further includes applying a further linear force to the sealed package to dispense the activated therapeutic substance fro the dispensing device. The administration assembly may include a housing and a compression member, wherein applying the linear force includes moving the compression member linearly relative to the housing.

An example method of dispensing a substance to a subject includes providing a dispensing device having a compression member, a housing, and a sealed package. The sealed package includes at least first and second sealed compartments that include different fluids. The method includes moving the compression member relative to the housing to apply a linear compression force to at least one of the first and second sealed compartments to merge the fluids of the first and second sealed compartments. The method further includes compressing at least one of the first and second sealed compartments to dispense the merged fluids to the subject. Compressing to dispense the merged fluids may include applying a linear compression force to at least one of the first and second sealed compartments.

Accordingly, the present disclosure has been described with some degree of particularity directed to the exemplary embodiments of the present disclosure. It should be appreciated, though, that the present disclosure is defined by the following claims construed in light of the prior art so that modifications or changes may be made to the exemplary embodiments of the present disclosure without departing from the inventive concepts contained herein.

What is claimed is:

1. A device for administering a therapeutic substance, comprising:
a sealed package including a collapsible compartment containing the therapeutic substance; and
an administration assembly comprising:
a dispenser in fluid communication with the therapeutic substance; and
at least one compression member movable by linear actuation to progressively collapse the collapsible compartment and urge the therapeutic substance through the dispenser.

2. The device of claim 1, wherein the sealed package includes at least first and second collapsible compartments, and the at least one compression member progressively collapses at least one of the first and second collapsible compartments to combine the therapeutic substances of the first and second collapsible compartments.

3. The device of claim 1, wherein the dispenser comprises a needle.

4. The device of claim 1, wherein the at least one compression member is movable by linear actuator in a first direction to collapse the collapsible compartment, and movable by linear actuation in a second direction to urge the therapeutic substance through the dispenser.

5. The device of claim 1, wherein the at least one compression member changes direction of motion between progressively collapsing the collapsible compartment and urging the therapeutic substance through the dispenser.

6. A single use dispenser for hypodermic administration of a unit dose of a therapeutic fluid to a subject, comprising:
a hermetically sealed package comprising:
a collapsible first compartment containing a first therapeutic substance and comprising a first flexible wall;
a collapsible second compartment containing a second therapeutic substance and comprising a second flexible wall;
a frangible seal disposed between the collapsible first and second compartments; an administration assembly comprising:
a dispenser positionable in fluid communication with at least one of the collapsible first and second compartments;
a compression member operable to rupture the frangible seal to permit the first and second therapeutic substances to merge and be dispensed.

7. The single use dispenser of claim 6, further comprising a rigid backing supporting the hermetically sealed package.

8. The single use dispenser of claim 6, wherein operation of the administration assembly moves the dispenser between extended and retracted positions.

9. The single use dispenser of claim 6, wherein the administration assembly includes an actuator linearly operable to move the compression member to force the merged first and second therapeutic substances through the dispenser.

10. The single use dispenser of claim 6, wherein the compression member comprises a roller.

11. The single use dispenser of claim 6, wherein the compression member is movable in opposite directions to first rupture the frangible seal and then force the first and second therapeutic substances through the dispenser.

12. The single use dispenser of claim 6, wherein the dispenser comprises an elongate needle.

13. The single use dispenser of claim 12, wherein the elongate needle extends in a direction perpendicular to a direction of movement of the compression member.

14. A therapeutic package for use in administering a dose of a therapeutic substance, comprising:
first and second sealed compartments mounted to a planar surface of a backing member and being pre-filled with first and second therapeutic substances, respectively;
a frangible seal disposed between the first and second sealed compartments;
a compression member operable to apply a first force to at least one of the first and second sealed compartments in a first direction in a plane parallel with the planar surface of the backing member to rupture the frangible seal and combine the first and second therapeutic substances to form a merged substance, and operable to apply a second force in a second direction opposite the first direction to force the merged substance out of the therapeutic package.

15. The therapeutic package of claim 14, wherein the first sealed compartment has a greater volume than the second sealed compartment.

16. The therapeutic package of claim 14, further comprising a housing and an actuator, the first and second sealed compartments being retained in the housing, and the compression member being coupled to the actuator, wherein advancing and retracting the actuator relative to the housing applies the first and second forces.

17. The therapeutic package of claim 14, wherein the compression member is coupled to a lever, and pivotal movement of the lever moves the compression member to apply the first and second forces.

18. The therapeutic package of claim 14, wherein the compression member comprises a planar portion arranged to contact at least one of the first and second sealed compartments to apply at least one of the first and second forces.

19. A method of administering a dose of a therapeutic substance, comprising:
providing a dispensing device having an administration assembly and a sealed package;
operating a portion of the administration assembly linearly to apply a first force to at least one sealed portion of the sealed package to activate a therapeutic substance contained therein;
operating the portion of the administration assembly linearly to apply a second force to the sealed package to dispense the therapeutic substance from the dispensing device.

20. The method of claim 19, wherein the administration assembly comprises a housing, a compression member, and an actuator, and operating the actuator linearly relative to the housing applies the first and second forces with the compression member.

21. The method of claim 20, wherein the administration assembly comprises a needle and an actuator, the method further comprising moving the needle out of the housing with operation of the actuator.

22. The method of claim 19, wherein applying the first force occurs in a first direction, and applying the second force occurs in a direction opposite the first direction.

23. A method of dispensing a substance to a subject, comprising:
providing a dispensing device having a compression member, a housing, and a sealed package, the sealed package including first and second sealed compartments that include different substances;
situating the dispensing device proximate an application region on the subject;
moving the compression member relative to the housing to apply a compression force to at least one of the first and second sealed compartments to combine the substances of the first and second sealed compartments and form a merged substance;
compressing at least one of the first and second sealed compartments to dispense the merged substance to the subject at the application region.

24. The method of claim 23, wherein the dispensing device includes an actuator coupled to the compression member, and compressing to dispense the merged substance includes moving the actuator linearly to apply a compression force to at least one of the first and second sealed compartments.

25. The method of claim 23, wherein the dispensing device includes an actuator coupled to the compression member and movable relative to the housing, the method including moving the actuator linearly relative to the housing to move the compression member relative to the housing to apply the compression force.

26. The method of claim 23, wherein the dispensing device includes an actuator coupled to the compression member and movable relative to the housing, the method including pivoting the actuator relative to the housing to move the compression member relative to the sealed package.

27. A dispensing device, comprising:
a sealed package comprising:
at least one compartment containing a substance;
a backing having at least one compression surface with a first surface shape when in a rest position; and
a compression panel for compressing the at least one compartment between the backing and the compression panel, the compression panel having at least one compression surface with a second surface shape when in a rest position that is different than the first surface shape;
wherein at least one of the first and second surface shapes is deformable to substantially accept a shape of the other during compression of the at least one compartment.

28. The dispensing device of claim 27, further comprising an actuator configured to cause dispensing of the substance from the dispensing device, wherein a position of the actuator before operation of the dispensing device and after dispensing the substance is substantially the same.

29. The dispensing device of claim 28, wherein the actuator causes mixing of at least two substances prior to dispensing.

30. A medical syringe for injecting a dose to a subject, comprising:
a dispensing assembly supporting a Luer lock connector that is configured to receive a needle, the Luer lock being retractable upon completion of injection of the dose to draw at least a portion of the needle into the medical syringe.

31. The medical syringe of claim 30, wherein the medical syringe further includes a sealed package that contains the dose.

32. The medical syringe of claim 30, wherein the dispensing assembly includes an actuator and a compression member, and linear operation of the actuator causes the compression member to inject the dose.

33. A sealed package, comprising:
a backing comprising an extension accommodating an dispensing feature;
at least one compartment mounted to the backing and containing at least one substance.

34. The sealed package of claim 33, wherein the backing includes a first surface upon which the at least one compartment is mounted, and the extension extends perpendicular to the first surface.

35. The sealed package of claim 33, further comprising a channel disposed on the backing and providing fluid communication between the at least one compartment and an administration feature.

36. The sealed package of claim 35, wherein a frangible seal is disposed along the channel to temporarily prevent fluid communication between the at least one compartment and the administration feature.

37. The sealed package of claim 33, wherein the dispensing feature is at least one of a needle, a mini needle, a micro-needle, a connector, a male Luer connector, a female Luer connector, a topical applicator, a spray nozzle, an ophthalmic applicator, an ear dispenser, a fitting, or a tube.

38. A dispensing device, comprising:
a compression member; and
a sealed package comprising a first frangible seal and a second frangible seal that at least partially define a perimeter of a first compartment;
wherein moving the compression member in a first direction ruptures the first frangible seal, and moving the compression member in a second direction ruptures the second frangible seal.

39. The dispensing device of claim 35, wherein the compression member has a panel construction.

40. The dispensing device of claim 35, wherein the sealed package includes first and second compartments containing first and second substances, respectively, the first frangible seal is defined between the first and second compartments, and rupturing the first frangible seal permits merging of the first and second substances.
